(19) United States
(12) Patent Application Publication
(10) Pub. No.: US 2023/0021329 A1
(43) Pub. Date: Jan. 26, 2023

(54) NANOSTRUCTURED BINARY GEL COMPOSITION AND USE THEREOF

(71) Applicant: Veri Nano Inc., Cambridge, MA (US)

(72) Inventors: Guillermo U. RUIZ ESPARZA HERRERA, Boston, MA (US); Kanny CHANG, Brighton, MA (US); Ketian ZHANG, Boston, MA (US); Samson AFEWERKI, Uppsala (SE)

(73) Assignee: Veri Nano Inc., Cambridge, MA (US)

(21) Appl. No.: 17/787,587

(22) PCT Filed: Dec. 18, 2020
(86) PCT No.: PCT/US2020/065994

§ 371 Date: Jun. 20, 2022

Related U.S. Application Data
(60) Provisional application No. 62/951,982, filed on Dec. 20, 2019.

(51) Int. Cl. A61K 31/722; A61K 9/06; A61K 9/16; A61K 47/02; A61K 38/38; A61K 47/32; A61K 47/10; A61K 49/00; A61P 31/04

(52) U.S. Cl. CPC A61K 31/722; A61K 9/06; A61K 9/1611; A61K 47/02; A61K 38/385; A61K 47/32; A61K 47/10; A61K 49/0041; A61P 31/04

(57) ABSTRACT

The present invention relates to a composition comprising a plurality of silicate nanoparticles; a hydrophilic polymer; and water, and optionally further comprising a thickening polymer; a plurality of zinc oxide particles; a polymer comprising a plurality of quaternary ammonium functional groups; an adhesive polymer; an oil or a polyol. The invention also relates to a use of the composition in prevention and/or treatment of a disease in a mammal, such as skin disease or mastitis. The invention relates further to a sealant of a duct in a body of a mammal comprising said composition.

### FIELD OF THE INVENTION

The present invention relates to a hydrogel composition comprising a plurality of silicate nanoparticles and a hydrophilic polymer for use in prevention and/or treatment of a disease in a mammal, such as skin disease or mastitis. The invention relates further to a sealant of a duct in a body of a mammal comprising said composition.

### BACKGROUND OF THE INVENTION

Microbial infections of wounds, tissues and organs can affect the physiological function of the tissue and the healing process. Microbial infections can lead to life-threatening complications.

In the field of animal disease, mastitis, a catastrophic mammary gland inflammatory process, represents the most common and costly disease for the dairy cattle industry in the United States.

Mastitis presents as a localized inflammatory reaction mediated by immune cells in response to the microbial invasion of the teat canal or as a result of a chemical, mechanical or thermal injury. In the case of microbial infections, toxins and proinflammatory cytokines are released and, as a result, the milk-secreting tissue and mammary ducts are damaged, causing mastitis. The chances of microbial infection are specially increased after milking, as the teat canal remains open for 15 minutes, which potentially exposes the duct to dirt, feces, urine, or other infectious agents. When an infectious agent is involved, transmission to other animals can occur after repetitive exposure to contaminated equipment and materials, or after use of contaminated gloves or hands.

Mastitis may be diagnosed by the observation of pathological changes in the glandular tissue of the udder, such as inflammation (heat, pain, redness, and swelling), odor, and ulcer.

As a result of this disease, changes in milk composition and appearance can be observed resulting in lower milk quality, for example, concentrations of calcium, casein, potassium, or lactoferrin may decline, and a waterier milk consistency with some aggregates can be observed. An increase of the somatic cell levels in the milk is used for diagnosis of mastitis.

Bacterial mastitis may be treated with antibiotics. However, milk from treated cows is not viable for human consumption until drug metabolites have been cleared from the cow's body. Although disease severity can be decreased by vaccination, this approach has proven to be ineffective in the long term, as it does not prevent mastitis due to the existence of multiple types of microbes.

Infections may, in large part, be prevented by using a sterile milking routine; the initial step in such a routine include applying iodine in the form of a spray to the teat and the surrounding areas, as a second step wiping and drying prior to milking, and as a third step applying the milking equipment to the teat canal. when milking is finished, the teats are carefully cleaned to prevent bacterial growth. Currently, several commercial products to prevent or treat mastitis exist, like ToMORROW®, Orbenin©, Orbeseal©, to name a few, and are intended to be applied after milking to form a physical barrier and block the entrance of the teat canal to prevent internal bacterial infiltration or to release antibiotics to treat the localized infection.

There exists a need for an antibacterial sealant for the treatment or prevention of microbial mastitis or for the treatment or prevention of a microbial infection of injuries, in humans and animals. There also exists a need for effective treatments of infected topical injuries, in humans and animals.

### SUMMARY OF THE INVENTION

It is an object of the present invention to at least partly overcome the above-mentioned problems, and to provide an improved composition for use in prevention and treatment of infections, especially mastitis in cows.

This object is achieved by a composition comprising or consists of
a plurality of silicate nanoparticles;
optionally, a plurality of zinc oxide particles;
a hydrophilic polymer;
a thickening polymer;
optionally, a polymer comprising a plurality of quaternary ammonium functional groups;
optionally, an adhesive polymer;
optionally, an oil or a polyol; and
water.

In some embodiments, the invention relates to any of the compositions described herein, wherein the pH of the composition is from 5 to 13. In certain aspects, the pH of the composition is from 7 to 13, or from 8 to 11.

According to an aspect of the invention, the composition comprises or consists of

a plurality of silicate nanoparticles;

a hydrophilic polymer; and water,

and optionally further comprising a thickening polymer; a plurality of zinc oxide particles; a polymer comprising a plurality of quaternary ammonium functional groups; an adhesive polymer; an oil or a polyol.

Because of the thickening properties of the silicate nanoparticles, the thickening polymer and the silicate nanoparticles may be the same ingredient.

The composition is biocompatible and biodegradable. The composition is non-toxic. Silicate nanoparticles or nanoplatelets are stable, safe, and non-irritable in vivo. They also exhibit anti-inflammatory properties. They are negatively charged on their surface and bear a positive charge on their edges. This unique charge distribution allows for surface-to-edge electrostatic interactions, which result in a three-dimensional “house-of-cards” structure. When combined with the other components of the composition, a physically reversible gel is formed. In certain aspects, if certain ions are present in the composition, they may electrostatically shield the interactions, thereby impairing the nanoplatelets from assembling. For example, pyrophosphates absorb onto the positively charged edge, thereby impairing formation of the three-dimensional structure. In certain aspects, this strategy can be employed to reverse gelation; the gel may be easily removed upon addition of a salt.

The compositions described herein provide advantages over the current sealants on the market, including, enhanced ease of use (both administration and removal The composition is elastic (pliable), The composition is mucoadhesive and having long-lasting tissue adherence. The composition is syringable (shear-thinning). The composition has a quick gelation time at about 37° C. The composition is inert. Furthermore, in some aspects, the sol-gel polymer compositions demonstrate a tailorable shear-thinning characteristic, which allows for ease of infusion over a wide temperature range as well as removal by manual stripping from the teat canal. Upon removal from the body cavity, the sol-gel polymer composition can return to a liquid phase at room temperature. In addition, unlike current commercial sealants, the compositions of this disclosure do not stick to the stainless-steel pipes (e.g., milk lines) or bulk tanks during the initial processing stage of milking and can readily be removed from industrial surfaces during standard cold or hot water washes, thereby reducing contamination of milk.

In some aspects, the silicate nanoparticles have a characteristic size of from about 1 nm to about 500 nm, for example, from about 10 nm to about 100 nm, or about 25 nm. In some aspects, the amount of silicate nanoparticles is from 0.1 wt % to 15 wt %, or from 3 wt % to 12 wt %, or about 10 wt %. In some aspects, the silicate nanoparticles are lithium magnesium sodium silicate nanoparticles.

In some aspects, the hydrophilic polymer is selected from the group comprising or consisting of poly(ethylene oxide), polyvinyl acetate, hydroxypropyl cellulose, and poloxamer. In some aspects, the hydrophilic polymer is poly(ethylene oxide). In some aspects, the poly(ethylene oxide) (PEO) has a molecular weight from about 1 kDa to about 10,000,000 kDa, or from about 15 kDa to about 25 kDa, or about 20 kDa. In some aspects, the amount of hydrophilic polymer or poly(ethylene oxide) is from 0.5 wt % to 10 wt %, or from 1 wt % to 3 wt %, or about 2 wt %. PEO is a biocompatible and FDA-approved hydrophilic polymer. When used as a film or a physical barrier, PEO works by means of a surface steric exclusion mechanism to exclude cells or fibrin from protected tissues.

Due to the properties of both the silicate nanoparticles and the hydrophilic polymer, such as adhesivity, gel-forming capabilities, as well as antimicrobial properties, a composition comprising or consisting of a plurality of silicate nanoparticles and a hydrophilic polymer can be obtained and applied for all uses mentioned herein. Such a composition is simple to obtain and manufacture. The costs for such a composition is relatively low. The composition is stable, biodegradable and non-toxic for all intended uses.

In some aspects, the thickening polymer is selected from the group comprising or consisting of silicate nanoparticles, silicate nanoparticles(Na_{0.7}[(Mg_{5.5}Li_{0.3})Si₈O₂₀(OH)₄]_{0.7}) (Laponite®), carboxymethyl cellulose, hydroxypropyl methylcellulose, hydroxyethyl cellulose, and agar, or derivatives thereof. In some aspects, the thickening polymer is carboxymethyl cellulose. In some aspects, a 2 wt % solution of the thickening polymer in water at 25° C. has a viscosity from about 200 mPa to about 2,000 mPa, or about 400 mPa to about 900 mPa. In some aspects, the amount of thickening agent or carboxymethyl cellulose is from 3 wt % to 9 wt % or from 4 wt % to 8 wt %, or about 6 wt %. The thickening agent may be silicate nanoparticles. In case an additional thickening agent is needed, these can easily be added to the composition to further improve the thickness of the composition. The thickening agent is easy to provide at low cost, biodegradable and non-toxic.

For instance, the nanosilicate Laponite® have previously been demonstrated to be biodegradable, which degrade to non-toxic products such as silicic acid (Si(OH)₄, sodium ions (Na⁺), magnesium ions (Mg²⁺) and lithium ions (Li⁺)) (Reference: H. Tomas, C. S. Alves, J. Rodrigues, Laponite®: A key nanoplatform for biomedical applications?, Nanomedicine: Nanotechnology, Biology, and Medicine 14 (2018) 2407-2420). The thickening agent carboxymethyl cellulose is also known to be biodegradable and non-toxic as have been presented previously (Reference: C. G. VanGinkel, S. Gayton, The biodegradability and nontoxicity of carboxymethyl cellulose (DS O.7) and intermediates, Environmental Toxicology and Chemistry, 1996, 15, 270-274). Furthermore, Laponite® and carboxymethyl cellulose have been widely employed for their thickening properties to improve the rheology properties of various formulations (Reference: F. R. Fitch, P. K. Jenness, S. E. Rangus, Rheological study of blends of Laponite and polymeric thickeners, Advances in Measurement and Control of Colloidal Processess, 1991, 292-307). The Laponite® is commercially available nanosilcate that can be obtained at low cost (Reference: B. Y. K. Additives, Laponite: Performance Additives, Wesel, Germany). The compositions of the invention demonstrated no cytotoxicity as shown in FIGS. 23A-23B and 22A-22B.

In some aspects, the polymer comprising a plurality of quaternary ammonium functional groups comprises or consisting of a plurality of repeat units having the following structure I:

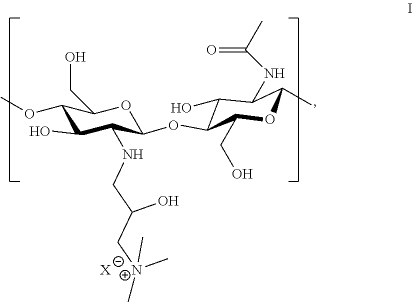

wherein X⁻ is selected from the group comprising or consisting of Cl⁻, Br⁻, BF₄⁻, and PF₆⁻. In some aspects, the polymer comprising a plurality of quaternary ammonium functional groups is selected from the group comprising or consisting of chitosan comprising a plurality of quaternary ammonium functional groups.

In some aspects, the amount of polymer comprising a plurality of quaternary ammonium functional groups is from 0.2 wt % to 2 wt %, or from 0.3 wt % to 10 wt %, or about 0.4 wt %.

Chitosan, when derivatized to include quaternary ammonium functional groups, may have antimicrobial and mucoadhesive properties. The polymer may improve the mucoadhesive properties of the composition. The polymer comprising a plurality of quaternary ammonium functional groups may thus further improve the antimicrobial or anti-infectious properties of the composition. In this context, it is well-known that quaternary ammonium functionalized chitosan displays improved antibacterial properties compared to their corresponding chitosan, among others due to the improved interaction with the negatively charged microbial cell membrane (R. C. Goy, D. de Britto, 0. B. G. Assis, A review of the antimicrobial activity of chitosan, Polímeros, 19, 3, 2009, 241-247; and H. Y. Atay, Antibacterial Activity of Chitosan-Based Systems. In: Jana S., (eds.) Functional Chitosan. Springer, Singapore, 2020, 457-489; and Afewerki, S. Bassous, N. Harb, S. Palo-Nieto, C. Ruiz-Esparza, G. U. Marciano, F. R. Webster, T. J. Lobo, A. O. Advances in antimicrobial and osteoinductive biomaterials, In Racing the surface (ed. B. Li et al.) Springer Nature, 2020, 3-34; and Afewerki, S. Bassous, N. Harb, S. Palo-Nieto, C. Ruiz-Esparza, G. U. Marciano, F. R. Webster, T. J. Furtado, A. S. A. Lobo, A. O. Advances in Dual Functional Antimicrobial and Osteoinductive Biomaterials for Orthopedic Applications, Nanomedicine: Nanotechnology, Biology, and Medicine, 2020, 14, 102143). Further advantages with the use of quaternary ammonium functionalized chitosan is their improved water solubility since chitosan is insoluble in water in neutral and alkaline aqueous (D. Zhu et al. Enanced water-solubility and antibacterial activity of novel chitosan derivatives modified with quaternary phophonium salt. Mater. Sci. Eng. C Mater. Biol. Appl. 2016, 1; 61:79-84). Chitosan and its derivatives have also shown to be a good candidate for the use in wound healing (K. Azuma et al. Chitin, Chitosan, and its Derivatives for Wound Healing: Old and New Materials. J. Funct. Biomater. 2015, 6(1): 104-142; and S. Ahmed, S. Ikram, Chitosan Based Scaffolds and Their Applications in Wound Healing. Achievements in Life Sciences, 2016, 10, 1, 27-37).

In some aspects, the amount of plurality of zinc oxide particles is from about 0.2 wt % to 5 wt %. Zinc oxide is an FDA-approved material and recognized as safe in food and topical use. Zink oxide may have antimicrobial and mucoadhesive properties. The zinc oxide may thus further improve the antimicrobial or anti-infectious properties of the composition. The use of zinc oxide in combination with quaternary ammonium functionalized chitosan will provide a synergistic antimicrobial activity since they have both different mechanism of action and also might target different microbials. (Afewerki, S. Bassous, N. Harb, S. Palo-Nieto, C. Ruiz-Esparza, G. U. Marciano, F. R. Webster, T. J. Lobo, A. O. Advances in antimicrobial and osteoinductive biomaterials, In Racing the surface (ed. B. Li et al.) Springer Nature, 2020, 3-34; and Afewerki, S. Bassous, N. Harb, S. Palo-Nieto, C. Ruiz-Esparza, G. U. Marciano, F. R. Webster, T. J. Furtado, A. S. A. Lobo, A. O. Advances in Dual Functional Antimicrobial and Osteoinductive Biomaterials for Orthopedic Applications, Nanomedicine: Nanotechnology, Biology, and Medicine, 2020, 14, 102143). For instance, ZnO is believed that the mechanism of action is through the generation reactive oxygen species (oxidative stress) or binding to the bacterial surface through electrostatic mechanism (Y. Xie, Y. He, P. L. Irwin, T. Jin, X. Shi, Antibacterial Activity and Mechanism of Action of Zinc Oxide Nanoparticles against Campylobacter jejuni, Appl. Environ. Microbiol. 2011, 2325-2331; and V. Tiwari et al. Mechanism of Anti-bacterial Activity of Zinc Oxide Nanoparticle Against Carbapenem-Resistant Acinetobacter baumannii, Front. Microbiol. 2018, 9, 1218.). The mechanism of quaternary ammonium functionalized chitosan is through electrostatic interaction between the polycation chitosan and anionic components in the microbials (H. Tan et al. Quaternized Chitosan as an Antimicrobial Agent: Antimicrobial Activity, Mechanism of Action and Biomedical Applications in Orthopedics, Int. J. Mol. Sci. 2013, 14, 1854-1869).

In some aspects, the adhesive polymer is poly(vinyl acetate). In some aspects, the amount of adhesive polymer or poly(vinyl acetate) is from 0.1 wt % to 50 wt %, or from 0.1 wt % to 5 wt %. Poly (vinyl acetate) is a non-toxic rubbery synthetic polymer with a molecular weight (Mw) ranging from 100,000 to 500,000. When used as an additive to a gel composition, the adhesive polymer may enhance tissue adhesiveness. The advantages of having optimal adhesive property of the composition will ensure that the gel stays in the place of applications e.g. tissue or in the teat canal preventing it from leaching or fall off from the place. For instance, in the application of preventing mastitis it should be able to stay in the teat canal intact without leaching during the entire dry period (120 days). The adhesive polymer poly(vinyl acetate) is compatible with the other ingredients in the composition and will induce adhesive property in composition without any negative impact on the property and performance of the gel.

In some aspects, the oil is selected from the group comprising or consisting of synthetic, mineral, plant and animal-based oils. In some aspects, the oil is selected from the group comprising or consisting of paraffin, coconut oil, olive oil, palm oil, soybean oil, canola oil (rapeseed oil), corn oil, peanut oil, and avocado oil. In some aspects, the amount of oil is about 20 wt % to about 80 wt %. In some aspects, the polyol is glycerol. In some aspects, the amount of polyol or glycerol is from 1 wt % to 80 wt %, or from 20 wt % to 70 wt %. Glycerol (or glycerin) may be used as an anti-freezing and, anti-drying agent to improve the long-term stability of the gel.

In some aspects, the composition is in the form a gel or binary gels. In some aspects, the gel has a density from 1.0 g/ml to 3.0 g/ml at about 38° C. In some aspects, the gel has a density from 1.1 g/ml to 5.0 g/ml or from 1.5 g/ml to 2.5 g/ml at about 38° C. The composition is capable of being spread (spreadable). This is important for the ability of the composition to be used in a syringe and for easily and evenly spreading of the composition on the skin of a subject or animal.

In some aspects, the sol-gel polymer composition has shear thinning properties such that the composition can be deformed in a syringe at room temperature. In certain aspects, the sol-gel polymer composition is capable of being injected using a single-barrel syringe and the like. In some aspects, the gel is injectable with injection force <100 N with tip a diameter of about 1.5 mm at an administration rate of about 2.5 ml/s. In certain aspects, the composition is shear-thinning.

In some aspects, the gel is stable in the presence of milk or water at 37-40° C. for up to about 120 days. In some aspects, the composition has mechanical strength from about 10² Pa to about 10⁵ Pa at strain value from about 10¹% to about 10³%. The advantage with a stable gel with the proper mechanical properties will ensure that the gel can resist the dynamic movement of the dairy cattle during the treatment. The unique composition in the gel makes it gain optimal mechanical property, which is stable in the environment of use. The composition has appropriate mechanical properties, for example, viscoelasticity that resembles the viscoelasticity of the tissue in the body to which the gel is applied, suggesting that the gel is able to withstand the constant dynamic/mechanical movement of the body. The physically crosslinking withing the gel composition in particular from the silica nanoparticles promote these properties. In some aspects, the composition comprises or consists of

1 to 15 wt % plurality of lithium magnesium sodium silicate nanoparticles;

0.5 to 10 wt % poly(ethylene oxide) having a molecular weight from about 1 kDa to about 10,000 kDa;

0.1 to 50 wt % polyvinylacetate;

20 to 70 wt % glycerol, and

up to 100 wt % water; and optionally further comprising

0.2 to 5 wt % plurality of zinc oxide particles and/or

0.2 to 2 wt % chitosan comprising a plurality of quaternary ammonium functional groups, wherein wt % are percentages of the total weight of the composition.

These concentrations would provide an optimal balance between the viability, antibacterial property and mechanical property of the composition. Too high concentrations of the ingredients might weaken the mechanical property of the composition and also have negative impact in the cytotoxicity. In some aspects, the composition as defined herein further comprises one or more drug or agent. These drug or agent may increase the usefulness or efficacy of the composition or facilitating detection. The drugs or agent can easily be incorporated in the composition by simply mixing the drug or agent with the other ingredients of the composition.

In some aspects, the one or more drug is selected from the group comprising or consisting of antibiotic drug, antiparasitic drugs, antimycotic drugs, analgesics or anti-inflammatory drugs, vitamins, corticosteroid drugs and proteins. Corporation of these drugs in the composition may have an additive or even synergistic effect on prevention and treatment of infections such as mastitis, skin wounds or any other disease or disorder mentioned herein. These drugs would have different mechanism of action than the antibacterial mode of action of the composition (mechanical barrier, quaternary chitosan and zinc oxide) and the combination of the composition and the drugs may allow treating and/or preventing a broad spectrum of microbials.

In some aspects, the one or more drug is selected from Rhodamine B or BSA Protein.

In some aspects, the one or more agent is selected from the group comprising or consisting of coloring agents or dyes and colorimetric pH indicators. The agents are useful as indicators on the presence of bacteria and the like. The agent may also be useful as indicators for replacement of the composition after administration, or as indicators for a quality of the composition. Having an indicator within the composition would simplify monitoring the performance of the composition and simplify for the stakeholder's in their application. In certain aspects, the composition is for medical and/or veterinary use. While human applications will become apparent from the disclosure, a preferred use relates to administration of the composition as defined herein to form a physical barrier in the teat canal of a dairy animal for the treatment and/or prevention of mammary diseases that may occur as the animal begins to dry off or during the dry period, comprising administering to the teat and/or within the teat canal of the animal the composition as defined herein.

The invention also relates to the composition as defined herein for use in prevention and/or treatment of a disease in a mammal. The invention relates to the composition as defined herein in the manufacture of a medicament for use in prevention and/or treatment of a disease in a mammal. The invention relates to a method of treating and/or preventing a disease, disorder or condition, which comprises administering to a mammal, such as a cow, in need thereof, a therapeutically effective amount of the composition as defined herein.

In some aspects, the composition as defined herein is for use in prevention and/or treatment of an infection in a mammal. In some aspects, the infection is a skin ulcer, a diabetic ulcer, acne, rosacea, an abscess, or an organ infection. In some aspects, the composition as defined herein is for use in prevention and/or treatment of a topical, subcutaneous, or internal infection. In some aspects, the composition as defined herein is for use in prevention and/or treatment of an inflammation in a mammal.

In some aspects, the composition is antimicrobial or bacteriostatic. In some aspects, the composition exhibits a MIC₅₀ of at least 40 μg/ml against S. aureus and/or E. coli. In some aspects, the composition as defined herein is for use in prevention and/or treatment of infections caused by S. aureus and/or E. coli.

In some aspects, the composition as defined herein is for use in prevention and/or treatment of mastitis/microbial mastitis in a mammal.

In some aspects, the composition as defined herein is for use in prevention and/or treatment of a skin disease in a mammal.

In some aspects, the composition as defined herein is for use in prevention and/or treatment of topical injuries, treatment of skin wounds, such as lacerations, abrasions, punctures, avulsions, ulcers and burns in an animal and or human through locally administering the composition.

In some aspects, the composition as defined herein is for use as a sealant/barrier of a duct in a body of a mammal, or as a teat sealant/barrier for a mammary duct. Some aspects relate to a sealant of a duct in a body of a mammal, or a teat sealant comprising or consisting of the composition as defined herein. In some aspects, the composition, upon administration, forms a physical barrier, thereby substantially impeding cell infiltration to the gel. In some aspects, the composition gels immediately upon administration/injection (for example, or in less than 60 seconds, or less than 30 second, or less than 10 seconds). The composition is thus believed to be capable of forming a no-leak, no-drip plug or durable seal after administration or injection into a mammalian subject.

In some aspects, the mammal is a dairy livestock animal, such as a heifer or a cow. In some aspects, the mammal is goats, sheep, horses, water buffaloes and the like. In some aspects, the mammal is a cow or a dairy cow.

In some aspects, the composition is administered inside a mammary duct and/or on a skin of an udder.

In some aspects, administering the sol-gel polymer composition as defined herein comprises applying topically the sol-gel polymer composition on mammary tissue, or applying within the teat canal (intra-mammarily). In some aspects, topical administration comprises dipping the teat in the composition. In some aspects, the composition is administered via intramammary administration, for example, by injection or infusion.

The invention relates to a method of administrating the composition as defined herein comprising or consisting of applying the composition applying within the teat canal (intra-mammarily) and/or topically.

In some aspects, the sol-gel polymer composition is administered when the animal begins to dry off or during the dry period of the animal. In certain aspects, the composition remains in the teat canal during the dry off period, thus reducing the clinical and sub-clinical cases of mastitis during the dry off period and in the first stage (post calving) of lactation. By remaining in the teat canal throughout the dry period, the sol-gel polymer composition minimizes microbial invasion through the teat canal during high risk periods in the pre-fresh dairy animal. In some aspects, the sol-gel polymer composition is administered prior to infection of a healthy animal. In some aspects, the sol-gel polymer composition is administered during the postpartum period of a non-lactating animal. In some other aspects, the sol-gel polymer composition is administered during the prepartum period of an animal.

The invention relates to a dosage regime for administering the composition as defined herein comprising or consisting of administered when the animal begins to dry off or during the dry period of the animal, and/or during the postpartum period and/or during the prepartum period.

In one aspect, milk obtained from the animal after administration of the composition as defined herein is used in the production of a milk product. Administration of the composition enables the reduction of withholding time of milk obtained from an animal being treated for mastitis, thereby decreasing the standard milk discard period that is required when the animals are given antibiotics.

In some aspects, the composition as defined herein is for use in delaying or preventing pregnancy. In some aspects, the composition as defined herein is for use in preventing pregnancy. By administering the composition as defined herein to the fallopian tubes of the mammal fluid communication between the ovary and the uterus can be impeded or prevented.

In some aspects, the invention relates to a kit comprising the compositions defined herein in a syringe, optionally with a manual for use of the syringe.

In some aspects, the invention relates to a kit comprising the compositions defined herein in a syringe, optionally with a manual for use of the syringe and a saline solution, optionally comprising 1-15 wt % tetrasodium pyrophosphate adapted fro removal of the composition from a surface or skin or udder or teat or teat canal. In some aspects, the low molecular weight glycol is ethylene glycol or polyethylene glycol with a molecular weight of 50 to 1000 g/mol.

### BRIEF DESCRIPTION OF THE DRAWINGS

The invention will now be explained more closely by the description of different embodiments of the invention and with reference to the appended figures.

FIG. 1 is a graph showing antibacterial properties of quaternary chitosan (qCh) at various concentrations against the Gram-positive Staphylococcus aureus (S. aureus) (circles) and Gram-negative Escherichia coli (E. coli) (squares). *MIC: minimum inhibitory concentration and MIC₅₀: minimum concentration that induced 50% growth reduction. *MIC of the antibiotic used in the commercial ToMORROW® sealant (cephapirin) against S. aureus and MIC₅₀ of cephapirin against E. coli are shown as a comparison.

FIG. 2A is a bar graph showing antibacterial properties of the hydrogel composition of the invention without 0.5 wt % qCh (Hydrogel only) and with 0.5 wt % qCh (0.5% qCh Hydrogel) (formulated as described in Example 2, with the following weight ratios: 100 g of water, 10 g of Laponite®, 8 g of carboxymethyl cellulose (CMC), 3 g of PEO, and with/without 0.5 g of qCh) against Gram-Positive S. aureus after 8 h.

FIG. 2B is a bar graph showing antibacterial properties of the hydrogel composition of the invention without 0.5 wt % qCh (Hydrogel only) and with 0.5 wt % qCh (0.5% qCh Hydrogel) (formulated as described in Example 2, with the following weight ratios: 100 g of water, 10 g of Laponite®, 8 g of carboxymethyl cellulose (CMC), 3 g of PEO, with/without 0.5 g of qCh) against Gram-Positive S. aureus after 24 h.

FIG. 3A is a bar graph showing antibacterial properties of the hydrogel composition of the invention without 0.5 wt % qCh (Hydrogel only) and with 0.5 wt % qCh (formulated as described in Example 2, with the following weight ratios: 100 g of water, 10 g of Laponite®, 8 g of carboxymethyl cellulose (CMC), 3 g of PEO, and with/without 0.5 g of qCh) against Gram-Negative E. coli after 8 h.

FIG. 3B is a bar graph showing antibacterial properties of Laponite® composition hydrogel without 0.5 wt % qCh hydrogel (Hydrogel only) and with 0.5 wt % qCh (formulated as described in Example 2, with the following weight ratios: 100 g of water, 10 g of Laponite®, 8 g of carboxymethyl cellulose (CMC), 3 g of PEO, and with/without 0.5 g of qCh) against Gram-Negative E. coli after 24 h.

FIG. 4A is a bar graph showing antibacterial properties of the hydrogel composition of the invention with 0.5 wt % qCh or with 1 wt % qCh, respectively against Gram-Negative E. coli after 8 h. Antibacterial efficacy of Orbeseal® is shown as a comparison group.

FIG. 4B is a bar graph showing antibacterial properties of the hydrogel composition of the invention with 0.5 wt % qCh or with 1 wt % qCh, respectively against Gram-Negative E. coli after 24 h. Antibacterial efficacy of Orbeseal® is shown as a comparison group.

FIG. 5 is a graph showing the results from strain sweep rheological experiments demonstrating storage (G′) and loss modulus (G″) versus oscillation strain of the hydrogel composition of the invention I (comprising 3% PEO (20 k molecular weight), 8% CMC and 15% Laponite) with 1.0 wt % qCh.

FIG. 6 is a graph showing the shear-thinning properties of the hydrogel composition of the invention with 0.5 wt % qCh (formulated as described in Example 2, with the following weight ratios: 100 g of water, 10 g of Laponite®, 8 g of carboxymethyl cellulose (CMC), 3 g of PEO, and 0.5 g of qCh) (viscosity versus shear stress). A decrease of viscosity at higher shear stress was observed, a typical behavior of shear-thinning biomaterials.

FIG. 7 is a graph comparing the recoverability of the hydrogel composition of the invention with 0.5 wt % qCh (formulated as described in Example 2, with the following weight ratios: 100 g of water, 10 g of Laponite®, 8 g of carboxymethyl cellulose (CMC), 3 g of PEO, and 0.5 g of qCh) and Orbeseal®. The resting state was set to 1% strain for 5 min and then high strain (100%) for 5 min was applied. Recovery of the formulations was monitored.

FIG. 8 is a graph showing injection force of the hydrogel composition of the invention with 0.5 wt % qCh (formulated as described in Example 2, with the following weight ratios: 100 g of water, 10 g of Laponite®, 8 g of carboxymethyl cellulose (CMC), 3 g of PEO, and 0.5 g of qCh). Acceptable injection force is <88 N.

FIG. 9A is a graph showing the long-term stability of the hydrogel composition of the invention without 0.5 wt % qCh hydrogel (Hydrogel only) (formulated as described in Example 2, with the following weight ratios: 100 g of water, 10 g of Laponite®, 8 g of carboxymethyl cellulose (CMC) and with 3 g of PEO, in PBS with or without 15 μg/mL lysozyme).

FIG. 9B is a graph showing the long-term stability of the hydrogel composition of the invention with 0.5 wt % qCh (formulated as described in Example 2, with the following weight ratios: 100 g of water, 10 g of Laponite®, 8 g of carboxymethyl cellulose (CMC), 3 g of PEO, and with 0.5 g of qCh) in PBS with or without 15 μg/mL lysozyme).

FIG. 10 is a graph showing the creation of the burn wound injury in porcine model as described in Example 14.

FIG. 11A is a graph showing the hydrogel composition of the invention #1 applied to the newly created burn wound injury in porcine model as described in Example 14.

FIG. 11B is a graph showing a form dressing applied direct contact with the hydrogel composition of the invention #1 as described in Example 14.

FIG. 11C is a graph showing the hydrogel composition of the invention #2 applied to the newly created burn wound injury in porcine model as described in Example 14.

FIG. 11D is a graph showing a form dressing applied direct contact with the hydrogel composition of the invention #2 as described in Example 14.

FIG. 11E is a graph showing the hydrogel composition of the invention #3 applied to the newly created burn wound injury in porcine model as described in Example 14.

FIG. 11F is a graph showing a form dressing applied direct contact with the hydrogel composition of the invention #3 as described in Example 14.

FIG. 12 is a graph showing after closing the burn injury wound in porcine model.

FIG. 13A is a graph showing the control experiments of the burn injury wound in porcine model at day 0.

FIG. 13B is a graph showing the control experiments of the burn injury wound in porcine model at day 7, showing the wounds had infections.

FIG. 14A is a graph showing the burn injury wound in porcine model treated with the hydrogel composition of the invention #1 at day 0.

FIG. 14B is a graph showing the burn injury wound in porcine model treated with the hydrogel composition of the invention #1 at day 7, showing no sign of any infection.

FIG. 14C is a graph showing the burn injury wound in porcine model treated with the hydrogel composition of the invention #2 at day 0.

FIG. 14D is a graph showing the burn injury wound in porcine model treated with the hydrogel composition of the invention #2 at day 7, showing no sign of any infection.

FIG. 14E is a graph showing the burn injury wound in porcine model treated with the hydrogel composition of the invention #3 at day 0.

FIG. 14F is a graph showing the burn injury wound in porcine model treated with the hydrogel composition of the invention #3 at day 7, showing no sign of any infection.

FIG. 15A is a table showing the amplitude sweep of the hydrogel composition of the invention #1-4 and the commercial product Orbeseal, performed as described in Example 7.

FIG. 15B is a graph showing a typical plot of the sweep oscillation strain curve of the hydrogel composition of the invention #4 described in Example 7.

FIG. 16A is a graph showing the recovery property of the hydrogel composition of the invention #1, performed in 7 cycles with high strain (400%) and low strain (0.1%) as described in Example 7.

FIG. 16B is a graph showing the recovery property of the hydrogel composition of the invention #2, performed in 7 cycles with high strain (400%) and low strain (0.1%) as described in Example 7.

FIG. 16C is a graph showing the recovery property of the hydrogel composition of the invention #3, performed in 7 cycles with high strain (400%) and low strain (0.1%) as described in Example 7.

FIG. 16D is a graph showing the recovery property the hydrogel composition of the invention #4, performed in 7 cycles with high strain (400%) and low strain (0.1%) as described in Example 7.

FIG. 16E is a graph showing the recovery property of gel from the commercial product Orbeseal, performed in 7 cycles with high strain (400%) and low strain (0.1%) as described in Example 7.

FIG. 17A is image showing the gel surface antibacterial tests (as Example 5 but with 100 μL bacterial solution (S. aureus) (instead of 20 μL) to fully cover the gel surface. Samples were shaken 120 rpm during incubation for normal bacterial growth and possible release of ingredients to the culture) of the hydrogel composition of the invention #1 and #4 (the gels were made with 300 kDa PEO as described in Example 13), and the commercial product Orbeseal (OB) at initial time point.

FIG. 17B is image showing the gel surface antibacterial tests (as Example 5 but with 100 μL bacterial solution (S. aureus) (instead of 20 μL) to fully cover the gel surface. Samples were shaken 120 rpm during incubation for normal bacterial growth and possible release of ingredients to the culture) of the hydrogel composition of the invention #1 and #4 (the hydrogel compositions were made with 300 kDa PEO as descried in Example 13), and the commercial product Orbeseal (OB) after 24 h of incubation.

FIG. 17C is bar graph showing antibacterial properties of the hydrogel composition of the invention #1 and #4 (the hydrogel compositions were made with 300 kDa PEO as descried in Example 13), the commercial product Orbeseal (OB) and 70% glycerin (as Example 5 but with 100 μL bacterial solution (S. aureus) (instead of 20 μL) to fully cover the gel surface. Samples were shaken 120 rpm during incubation for normal bacterial growth and possible release of ingredients to the culture) against Gram-Positive S. aureus after 24 h.

FIG. 17D is bar graph showing antibacterial properties of the hydrogel composition of the invention #1 and #4 (the hydrogel compositions were made with 300 kDa PEO as descried in Example 13), the commercial product Orbeseal (OB) and 70% glycerin (as Example 5 but with 100 μL bacterial solution (S. aureus) (instead of 20 μL) to fully cover the gel surface. The samples were not shaken which might result in preventing the possible release of ingredients to the culture) against Gram-Negative E. coli after 24 h.

FIG. 18A is graph showing antibacterial properties of the hydrogel composition of the invention #4 and the commercial product Orbeseal against Gram-Negative E. Coli 9 days of incubation, and the level of bacterial contamination was determined by optical density measurement at the wavelength of 600 nm, and photos showing the bacterial plating results of both the hydrogel composition of the invention #4 and the commercial product Orbeseal. Demonstrating higher and longer antibacterial efficacy compared to the commercial Orbeseal.

FIG. 18B is graph showing antibacterial properties of the hydrogel composition of the invention #4 and the commercial product Orbeseal against Gram-Positive S. aureus 9 days of incubation, and the level of bacterial contamination was determined by optical density measurement at the wavelength of 600 nm, and photos showing the bacterial plating results of both the hydrogel composition of the invention #4 and the commercial product Orbeseal. Demonstrating higher and longer antibacterial efficacy compared to the commercial Orbeseal.

FIG. 18C is bar graph showing prevention of bacterial invation after 14 days (antibacterial properties) of the hydrogel composition of the invention #4 and the commercial product Orbeseal against both Gram-Negative E. Coli and Gram-Positive S. aureus 9 days of incubation, Demonstrating the antibacterial efficacy as highly efficient in killing bacteria without the use of any antibiotics compared to the commercial Orbeseal.

FIG. 19A is bar graph showing antibacterial properties of the hydrogel composition of the invention #1, #2, #3 and #4 (the hydrogel compositions were made with 8, 000,000 kDa PEO as described in Example 13) and the commercial product Orbeseal (OB) (as Example 5) against Gram-Positive S. aureus (10⁸ CFU/mL) after 24 h.

FIG. 19B is bar graph showing antibacterial properties of the hydrogel composition of the invention #1 and #4 (the hydrogel compositions were made with 300 kDa PEO as described in Example 13) and the commercial product Orbeseal (OB) (as Example 5) against Gram-Positive S. aureus (10⁸ CFU/mL) after 24 h.

FIG. 20A is image showing the control sample of the fluorescence detection of ZnO in cells.

FIG. 20B is image showing the fluorescence detection of ZnO in cells after 5 min of incubation with 0.1% ZnO.

FIG. 20C is image showing the fluorescence detection of ZnO in cells after 30 min of incubation with 0.1% ZnO.

FIG. 20D is image showing the fluorescence detection of ZnO in cells after 60 min of incubation with 0.1% ZnO.

FIG. 21 is a graph showing injection force of the hydrogel composition of the invention #1 and #4 (the hydrogel compositions were made with 300 kDa and 8, 000,000 kDa PEO as described in Example 13) and the commercial product Orbeseal (OB)

FIG. 22A is a graph showing the cytotoxic (fibroblast cells were incubated 1 day after cell seeding. Cells (50 μL, 10⁵ CFU/mL seeding density) was added to 0.1 mL of the respective sample (the hydrogel composition #4 and Orbeseal) covered on a Transwell insert (3 μm pore size). The co-culture experiment was conducted with gentle shaking 5% CO₂ in a close chamber) results of the co-culture system of bacteria and cells.

FIG. 22B is a graph showing the cytotoxic results (performed as described in Example 15) of hydrogel composition #4 with dose escalation. Demonstrating no significant toxicity.

FIG. 23A is a graph showing the cytotoxic results (performed as described in Example 15) of the hydrogel composition #4 fabricated with 300 kDa PEO and 8, 000,000 kDa PEO as descried in Example 13 with mass escalation. Demonstrating no significant toxicity and no significant differences.

FIG. 23B is a bar graph showing the cytotoxic results (performed as described in Example 15) of 10% Laponite, 15% Laponite and Orbeseal (OB).

FIG. 24 is image showing the adhesive property of the hydrogel compositions by placing the gels between the researcher's fingers.

FIG. 25 is a graph showing the clinical results (as described in Example 18) based on somatic cell count (number of inflammatory cells in the milk) from the hydrogel composition of the invention #3 and #4. The trial demonstrated the hydrogel compositions stability in teat cannel during the entire dry period of 60 days with no sign of degradation or leakage. No mastitis was observed from the clinical trials with the hydrogel compositions #3 and #4.

FIG. 26 is a table showing elemental analysis of the milk and blood from the clinical results (as described in Example 18) based on the element Lithium (Li) from the hydrogel compositions #3 and #4.

FIG. 27 is absorbance spectra demonstrating the determination of the release of BSA from the hydrogel compositions (#4) using Nanodrop spectrophotometer (presented in Examples 19-20)FIG. 28 is calibration curve of Rhodamine B in PBS used for the determination of the release of Rhodamine B from the hydrogel composition (#4).

FIG. 29 is a table showing the clinical results (as described in Example 18) based on observations from the hydrogel compositions #3 and #4. The trial demonstrated the no sign of any mastitis and also normal appearance off all the cows treated.

Detailed Description of Various Embodiments of the Invention

### Definitions

Unless otherwise defined herein, scientific and technical terms used in this application shall have the meanings that are commonly understood by those of ordinary skill in the art. The following definitions are given merely to illustrate the general meanings of the main terms used in connection with the disclosure.

The term “wt %” as used herein means percentages of the total weight of the composition.

As used herein, the term “subject” shall include humans and terrestrial animals. For example, the subject can be a domestic livestock species, a laboratory animal species, a zoo animal, a companion animal or a human. In a particular aspect, “subject” refers more specifically to any lactating animal. The subject may be a cow.

The term “effective amount” is intended to qualify the amount of the composition that will achieve the goal of treating or preventing the disease or condition. “Effective” may also refer to improvement in disease severity or the frequency of incidence over no treatment.

The term “disease” is intended to include disorder, condition or any equivalent thereof.

The term “topical” shall refer to any composition applied to the epidermis. Topical shall also refer to compositions used as mouthwashes.

The terms “teat dip” or “teat dipping” shall be interpreted broadly and in accordance with the terminology used in the art of dairy farming. Thus, the composition is not only intended for dipping of the teats and can be applied in other ways, such as by spraying, which falls within the recognized terms teat dip or teat dipping composition or agent.

As used herein unless otherwise specified, the term “antimicrobial” describes a biocidal effect, such as killing or inhibiting the growth of microbes, that may have, for example, an antibacterial, antifungal, antiviral, bacteriostatic, antiprotozoal, disinfecting, or sanitizing effect.

As used herein unless otherwise specified, the term “microbes” shall be interpreted broadly and includes of pathogenic microorganisms such as bacteria, fungi, yeast, viruses, protozoans and the like.

The term “udder” refers herein to the glandular, mammary structure of a female ruminant animal such as a cow, a goat, a sheep, a water buffalo and the like. In the cow, it comprises four independent glands, with one teat and one exit duct each, whereas sheep and goat have two glands. The term “teat” refers herein to the projecting part of the mammary gland containing part of the milk or teat sinus.

The term “teat sealant” refers herein to compositions and devices used to form a physical barrier on the surface of or inside an animal teat. A teat sealant can be on the teat surface, inside the teat streak canal, and/or inside the teat cistern.

The term “solution” refers herein to solutions, suspensions, or dispersions, unless otherwise stated.

The term “spray” as used herein refers to an atomized composition, such as comprised of small or large liquid droplets, such as applied through an aerosol applicator or pump spray applicator for the intended purpose of delivering the composition over an area, such as skin around the teats.

The term “infusion” refers herein to the continuous introduction of a fluid or solution into a cavity, vein or cistern.

The term “mammal” refers herein to a warm-blooded vertebrate animal of the class Mammalia, which includes both human and animal, that possess hair or fur on the skin, the secrete milk from milk-producing mammary glands by females for nourishing the young, and a four-chambered heart.

For the aspects of the disclosure that relate to mastitis, the term “animal” refers herein to a female, non-human mammal which has a lactation period, which includes, but is not limited to, livestock animals, such as cows, sheep, goats, horses, pigs, water buffaloes and the like. Preferably, the animal is a dairy cow. While both the “cow” and the “heifer” are female bovines, the term “heifer” refers herein to any young female cow that has not given birth to a calf, typically one that has been weaned and under the age of 3 years. The term “cow” often refers to an older female animal that has given birth to a calf.

The term “dry period” refers herein to the non-lactating phase of the lactation cycle of a cow or other dairy animal. It occurs between the end of one lactation cycle and the beginning of the next lactation. At the end of each lactation cycle, the animal begins the phase of “drying off” as the animal enters the dry period, which includes the usual physiological, metabolic and endocrine changes associated with cessation of milk production for the non-lactating period (dry period) of the animal.

The term “milk product” refers herein to a product containing any amount of milk in liquid or powder form. It also includes cheese and yogurt.

The term “postpartum” refers herein to the period of time beginning immediately after calving and extending for about six weeks. The term “prepartum” refers herein to the period of time during pregnancy, which is prior to calving. The term “periparturient” refers herein to the period immediately before and after calving.

The term “involution” refers herein to the first two to three weeks after cessation of milk production in a cow.

The term “withdrawal period for milk” or “withholding time for milk” means the interval between the last administration of a veterinary medicinal product to animals under normal conditions of use and the production of milk from such animals to ensure that such milk does not contain residues in quantities in excess of the maximum residue limits established by regulatory authorities.

The term “microbial invasion” refers herein to movement of pathogenic microorganisms such as, for example, bacteria, especially pus-forming or necrotizing bacteria, viruses, fungi, yeast, protozoans and the like that proliferate into bodily tissue or bodily cavities, resulting in tissue injury that can progress to infection and/or disease. The “microbial invasion” may refer to a “bacterial invasion.”

The term “sol-gel polymer composition” refers herein to a polymer composition that can undergo a sol-gel process to form a sol-gel state under certain conditions, as described herein.

The term “polymer” refers herein to a material that includes a set of macromolecules. Macromolecules included in a polymer can be the same or can be differ from one another in some fashion. A macromolecule can have any of a variety of skeletal structures and can include one or more types of monomeric units. In particular, a macromolecule can have a skeletal structure that is linear or non-linear. Examples of non-linear skeletal structures include branched skeletal structures, such those that are star-branched, comb-branched, or dendritic-branched, and network skeletal structures. A macromolecule included in a homopolymer typically includes one type of monomeric unit, while a macromolecule included in a copolymer typically includes two or more types of monomeric units. Examples of copolymers include statistical copolymers, random copolymers, alternating copolymers, periodic copolymers, block copolymers, radial copolymers, and graft copolymers.

As used herein with reference to a polymer, the term “molecular weight (MW)” refers to a number average molecular weight, a weight average molecular weight, or a melt index of the polymer.

The term “elastic modulus” (also referred to as “Young's modulus” or the storage modulus (G′)) is defined herein as the change in stress with an applied strain (that is, the ratio of shear stress (force per unit area) to the shear strain (proportional deformation)) in a material. Essentially, the elastic modulus is a quantitative measurement of stiffness of an elastic material that measures the ability of the tested material to return to its original shape and size. G′ can be calculated using a formula derived from Hooke's law, which states that the elastic modulus is equal to the ratio of stress to strain (i.e. the ratio of applied pressure to fractional change in size). The measure of the elastic modulus is reported as the force per unit area (the standard metric ratio of the Newton to unit area (N/m²) or the pascal (Pa) in which one pascal is equivalent to one Newton (1 N) of force applied over an area of one meter squared (1 m²)). This pascal unit is an art-recognized term often used to define a unit of pressure, tensile strength, stress and elasticity.

As used herein, the term “drug” refers to any agent capable of having a physiologic effect (e.g. a therapeutic or prophylactic effect) on a biosystem such as prokaryotic or eukaryotic cells or organisms, in vivo or in vitro. The drug can be selected from a variety of known classes of drugs, including, for example, analgesics, anesthetics, anti-inflammatory agents, anthelmintics, anti-arrhythmic agents, antiasthma agents, antibiotics (including penicillin), anticancer agents (including Taxol), anticoagulants, antidepressants, antidiabetic agents, antiepileptics, antihistamines, antitussives, antihypertensive agents, antimuscarinic agents, antimycobacterial agents, antineoplastic agents, antioxidant agents, antipyretics, immunosuppressants, immunostimulants, antithyroid agents, antiviral agents, anxiolytic sedatives (hypnotics and neuroleptics), astringents, bacteriostatic agents, beta-adrenoceptor blocking agents, blood products and substitutes, bronchodilators, buffering agents, cardiac inotropic agents, chemotherapeutics, contrast media, corticosteroids, cough suppressants (expectorants and mucolytics), diuretics, dopaminergics (antiparkinsonian agents), free radical scavenging agents, growth factors, haemostatics, immunological agents, lipid regulating agents, muscle relaxants, proteins, peptides and polypeptides, parasympathomimetics, parathyroid calcitonin and biphosphonates, prostaglandins, radio-pharmaceuticals, hormones, sex hormones (including steroids), time release binders, anti-allergic agents, stimulants and anoretics, steroids, sympathomimetics, thyroid agents, vaccines, vasodilators, proteins, antibodies, and xanthines.

As used herein, the term “protein” refers to any large biomolecules and macromolecules that contain at least one polypeptide chain. The protein of interest can be selected from various proteins with different functions, including enzymes, antibodies, transmembrane proteins, cell signaling proteins (including insulin), and structural proteins (including collagen).

The term “shear thinning” as used herein refers to the common characteristic of non-Newtonian fluids in which the fluid viscosity decreases with increasing shear rate or stress. Shear thinning is observed in suspensions, emulsions, polymer solutions and gels. Due to shear thinning attributes, decreasing the viscosity of a polymer, a macromolecule or gel is made possible by increasing the rate of shear. Basically, as a result of the decrease in viscosity upon increase in shear rate, the “shear thinning” property is a measure of the ability of the gel network to be temporarily deformed through the application of a gentle manual pressure from the piston of a syringe. This shear thinning phenomenon may be used, for instance, to make an otherwise stiff biocompatible gel infusible.

The term “durable” as used herein means that a material does not exhibit a significant loss of mass upon exposure to conditions for a period of time, such as exposure to a buffered solution at physiological temperature for a period of time. For example, the material does not lose more than 10% of its original mass after exposure to a buffered solution at physiological temperature for about 4 weeks.

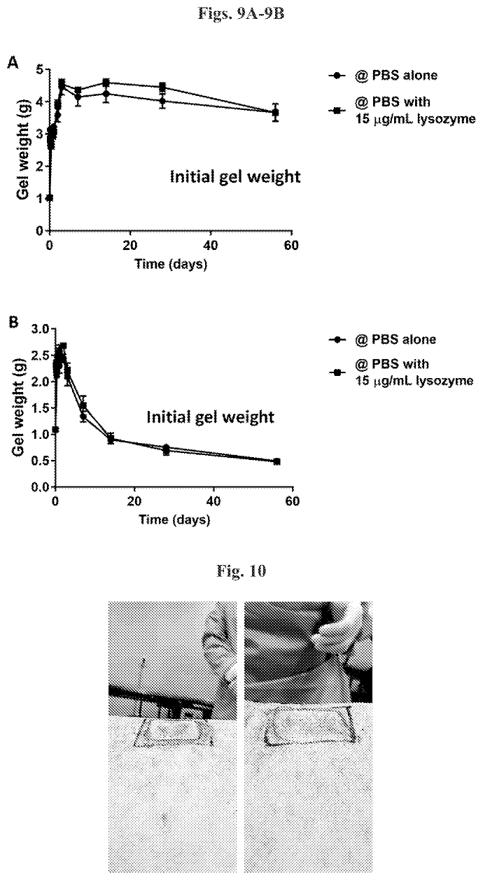

The term “stable” as used herein means that a material does not exhibit phase separation, as determined by differential scanning calorimetry (DSC), upon exposure to conditions for a period of time. Stability may be assessed by performing stability testing, for example, in PBS and in PBS/lysozyme presented in FIG. 9A and FIG. 9B.

The term “loss tangent tan δ” or “tan δ” refers herein to the tangent of the phase angle, that is, the ratio of viscous modulus (G″) to elastic modulus (G′) and a helpful quantifier of the presence and the degree of elasticity in a fluid. The tan δ values of less than unity indicate elastic-dominant (i.e. solid-like) behavior and values greater than unity indicate viscous-dominant (i.e. liquid-like) behavior. In an elastic solid, tan δ″=0.

As used herein, “strong” is intended to mean the elastic modulus G′ that can generally range widely from about 420 Pa or higher, or from about 600 Pa to about 10,000 Pa, or from about 6000 Pa to about 10,000 Pa, etc. at physiological temperature. Based on the level of stiffness, a solid body, for example, deforms when a load is applied to it. If the material is elastic, the body returns to its original shape after the load is removed. A “strong solid” is generally a gel or solid formed after the sol-gel phase transition for which G′ at physiological conditions (e.g., 37° C., and/or near physiological pH) is typically above about 560 Pa, although strong solids may form below about 560 Pa or above about 10,000 Pa depending on other factors in the processing steps to make, to sterilize or to store the composition.

The term “physiological temperature” used herein is intended to mean the normal body temperature range for a mammal, e.g., about 35° C. to about 40° C., about 36° C. to about 40° C., about 37° C., about 37.5° C. and the like.

The term “one or more physiological stimulus” refers herein to a selection of one or more stimulus embracing, but not limited to, temperature (e.g., body temperature such as a temperature from about 36° C. to about 40° C., or about 37° C.), pH (e.g., near physiological pH, alkaline or acidic conditions), ionic strength (e.g., hypotonic or hypertonic conditions) and the like. Other types of physiological stimuli include exposure to a bodily fluid such as, for example, breast milk or other secretions, blood, and the like. Another type of stimuli may arise from contact with a bodily chemical or macromolecule such as without limitation ions, electrolytes, calcium, sodium, cytotoxins, macrophages, enzymes, antigens, glucose, estrogen, etc.

As used herein, the term “characteristic size” as determined by transmission electron microscopy (TEM) means characteristic diameter, or, for a plurality of particles, mean, median, or mode diameter. In some aspects, “characteristic size” for a plurality of particles means that at least about 50%, at least about 60%, at least about 70%, at least about 80%, or at least about 90% of the particles have the recited characteristic size.

The term “hydrophilic,” as used herein, refers to a compound that has an octanol/water partition coefficient (K_{ow}) less than about 10 at about 23° C.

As used herein, a therapeutic that “prevents” a disease or condition refers to a compound or composition that, in a statistical sample, reduces the occurrence of the disease, disorder or condition in the treated sample relative to an untreated control sample, or delays the onset or reduces the severity of one or more symptoms of the disease, disorder or condition relative to the untreated control sample.

The term “treating” is art-recognized and includes ameliorating the disease, disorder or condition (i.e., arresting the disease or reducing the manifestation, extent, or severity of at least one of the clinical symptoms thereof). In another aspect “treating” or “treatment” refers to ameliorating at least one physical parameter, which may not be discernible by the subject. In yet another aspect, “treating” or “treatment” refers to modulating the disease, disorder or condition, either physically, (e.g., stabilization of a discernible symptom), physiologically, (e.g., stabilization of a physical parameter), or both. In a further aspect, “treating” or “treatment” relates to slowing the progression of the disease.

In certain aspects, the gel sealant comprises a combination of aqueous media and an organic liquid (such as glycerol).

In some aspects, the sol-gel polymer composition has viscoelastic and recoverability properties. For example, when 100% strain is applied to the gel (outside the viscoelastic region of the gel) for 5 min, the gel deforms. After removal of 100% strain, 1% strain is applied for 5 min, and the gel quickly recovers to its original structure, thus suggesting no permanent deformation of the hydrogel.

Composition

The invention relates to a composition, comprising
a plurality of silicate nanoparticles;
optionally, a plurality of zinc oxide particles;
a hydrophilic polymer;
a thickening polymer;
optionally, a polymer comprising a plurality of quaternary ammonium functional groups;
optionally, an adhesive polymer;
optionally, an oil or a polyol; and
water.

The silicate nanoparticles may also be the thickening polymer in which case the composition may comprise a plurality of silicate nanoparticles; a hydrophilic polymer; and optionally further comprising a thickening polymer; a plurality of zinc oxide particles; a polymer comprising a plurality of quaternary ammonium functional groups; an adhesive polymer; an oil or a polyol; and water.

The pH of the composition may be pH from 5 to 13, or from 6 to 13, or from 8 to 12, from 9 to 11.

The silicate nanoparticles may be any kind of silicate particles. The silicate nanoparticles may be lithium magnesium sodium silicate nanoparticles. The size or diameter of the particles may be from 1 nm to 500 nm, or from 10 nm to 100 nm, or from 10 nm to 50 nm, or from 15 nm to 30 nm, or about 25 nm.

The amounts in which the silicate nanoparticles is used in the composition may vary from 0.1 wt % to 20 wt %, or 5 wt % to 20 wt %, or 5 wt % to 15 wt %, or 7 wt % to 15 wt %,

A wide range of hydrophilic polymers may be used in the composition. Some, none limiting examples are hydrophilic polymer may be selected from the group comprising or consisting of poly(ethylene oxide), polyvinyl acetate, hydroxypropyl cellulose, and poloxamer. One or more hydrophilic polymers may be used. One hydrophilic polymer may be used having the same or different molecular weight. For example, poly(ethylene oxide) (PEO) may be used in a combination of two PEOs having a molecular weight of e.g. 20 k Da and 300 kDa. Having these combinations would provide PEO with different mechanical, adhesive and solubility property. The higher molecular weight of PEO provides improved mechanical strength and adhesion property, and more viscous. Moreover, PEO also prevent cell infiltration.

Or one PEO may be use having a molecular weight of e.g. 20 k Da. The amounts in which the hydrophilic polymer is used in the composition may vary from 0.5 wt % to 10 wt %, or from 1 to 7 wt % or from 2 to 6 wt %, or from 2 to 5 wt %.

A wide range of thickening polymer may be used in the composition. The thickening polymer may be silicate nanoparticles. Other examples of thickening polymer may be carboxymethyl cellulose, hydroxypropyl methylcellulose, hydroxyethyl cellulose, and agar, or derivatives thereof. One or more thickening polymer may be used. The thickening polymer may have a viscosity from about 200 mPa to about 2,000 mPa, preferably from about 400 mPa to about 900 mPa, in a 2 wt % solution of the thickening polymer in water at 25° C. The thickening polymer may be carboxymethyl cellulose (CMC), or CMC sodium salt having a viscosity of 500-900 mPa at 2% in water at 25° C.

The amounts in which the thickening polymer is used in the composition may vary from 3 wt % to 9 wt %, or 3.5 wt % to 7.5 wt %, or from 4 wt % to 8 wt %, or from 5 wt % to 7 wt %.

The plurality of quaternary ammonium functional groups may be a plurality of repeat units having structure formula I:

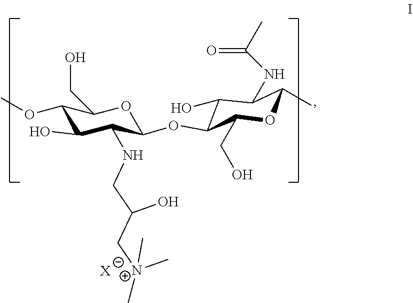

wherein X⁻ may be selected from the group comprising or consisting of Cl⁻, Br⁻, BF₄⁻, and PF₆⁻. X may be Cl.

The structural compound of formula I may be chitosan comprising a plurality of quaternary ammonium functional groups.

The amounts in which the quaternary ammonium compound is used in the composition may vary from 0.1 wt % to 10 wt %, or 0.2 wt % to 5 wt %, or from 0.2 wt % to 1 wt %, or from 0.2 wt % to 0.9 wt %, 0.4 wt % to 0.8 wt %, or about 0.5 et %.

A wide range of adhesive polymer may be used in the composition. One or more adhesive polymer may be used. Examples of adhesive polymers may be poly(vinyl acetete). This polymer is compatible with the other ingredients of the composition.

The amounts in which the adhesive polymer is used in the composition may vary from 0.1 wt % to 50 wt %, or from 0.1 wt % to 10 wt %, or 0.1 wt % to 5 wt %, 0.1 wt % to 3 wt %, 0.5 wt % to 2.5 wt %.

The amounts in which the plurality of zinc oxide particles is used in the composition may vary from 0.1 wt % to 5 wt %, or from 0.2 wt % to 1 wt %, or from 0.2 wt % to 0.75 wt %, or form 0.25 wt % to 5.5 wt %.

A wide range of oils and polyols may be used in the composition. Examples of oils may be synthetic, mineral, plant and animal-based oils, for example, paraffin, coconut oil, olive oil, palm oil, soybean oil, canola oil (rapeseed oil), corn oil, peanut oil, or avocado oil. The amounts in which the oil is used in the composition may vary from 20 wt % to 80 wt %, or from 30 wt % to 60 wt %.

Examples of polyols may be glycerol. Polyols may add several advantages and additional properties to the gel compositions such as anti-freezing, anti-drying and antibacterial properties.

The amounts in which the polyol or glycerol is used in the composition may vary from 1 wt % to 80 wt %, or from 20 wt % to 70 wt %, or from 25 wt % to 55 wt %, or from 30 wt % to 50 wt %.

The composition of the invention may comprise or consist of water, a plurality of silicate nanoparticles (e.g., Laponite® XLG), a hydrophilic polymer (e.g., poly(ethylene oxide) (PEO)), and a thickening polymer (e.g., carboxymethyl cellulose (CMC)).

The composition of the invention may comprise or consist of water, glycerol, a plurality of silicate nanoparticles (e.g., Laponite® XLG), a hydrophilic polymer (e.g., poly(ethylene oxide) (PEO)), a thickening polymer (e.g., carboxymethyl cellulose (CMC).

The composition of the invention may comprise or consist of water, a plurality of silicate nanoparticles (e.g., Laponite® XLG), a hydrophilic polymer (e.g., poly(ethylene oxide) (PEO)), a thickening polymer (e.g., carboxymethyl cellulose (CMC), and an adhesive polymer (e.g. poly(vinyl acetate)).

The composition of the invention may comprise or consist of water, glycerol, a plurality of silicate nanoparticles (e.g., Laponite® XLG), a hydrophilic polymer (e.g., poly(ethylene oxide) (PEO)), a thickening polymer (e.g., carboxymethyl cellulose (CMC), and an adhesive polymer (e.g. poly(vinyl acetate)).

The composition of the invention may comprise or consist of water, a plurality of silicate nanoparticles (e.g., Laponite® XLG), a hydrophilic polymer (e.g., poly(ethylene oxide) (PEO)), a thickening polymer (e.g., carboxymethyl cellulose (CMC)), and a polymer comprising a plurality of quaternary ammonium functional groups (e.g., chitosan functionalized with quaternary ammonium functional groups).

The composition of the invention may comprise or consist of water, glycerol, a plurality of silicate nanoparticles (e.g., Laponite® XLG), a hydrophilic polymer (e.g., poly(ethylene oxide) (PEO)), a thickening polymer (e.g., carboxymethyl cellulose (CMC)), and a polymer comprising a plurality of quaternary ammonium functional groups (e.g., chitosan functionalized with quaternary ammonium functional groups).

The composition of the invention may comprise or consist of water, a plurality of silicate nanoparticles (e.g., Laponite® XLG), a hydrophilic polymer (e.g., poly(ethylene oxide) (PEO)), a thickening polymer (e.g., carboxymethyl cellulose (CMC), a polymer comprising a plurality of quaternary ammonium functional groups (e.g., chitosan functionalized with quaternary ammonium functional groups) and an adhesive polymer (e.g. poly(vinyl acetate)).

The composition of the invention may comprise or consist of water, glycerol, a plurality of silicate nanoparticles (e.g., Laponite® XLG), a hydrophilic polymer (e.g., poly(ethylene oxide) (PEO)), a thickening polymer (e.g., carboxymethyl cellulose (CMC), a polymer comprising a plurality of quaternary ammonium functional groups (e.g., chitosan functionalized with quaternary ammonium functional groups) and an adhesive polymer (e.g. poly(vinyl acetate)).

The composition of the invention may comprise or consist of water, a plurality of silicate nanoparticles (e.g., Laponite®), a hydrophilic polymer (e.g., poly(ethylene oxide) (PEO)), a thickening polymer (e.g., CMC), and a plurality of oxidized metal particles (e.g., zinc oxide particles).

The composition of the invention may comprise or consist of water, glycerol, a plurality of silicate nanoparticles (e.g., Laponite®), a hydrophilic polymer (e.g., poly(ethylene oxide) (PEO)), a thickening polymer (e.g., CMC), and a plurality of oxidized metal particles (e.g., zinc oxide particles).

The composition of the invention may comprise or consist of water, glycerol, a plurality of silicate nanoparticles (e.g., Laponite®), a hydrophilic polymer (e.g., poly(ethylene oxide) (PEO)), a thickening polymer (e.g., CMC), a plurality of oxidized metal particles (e.g., zinc oxide particles), and an adhesive polymer (e.g. poly(vinyl acetate)).

The composition of the invention may comprise or consist of water, a plurality of silicate nanoparticles (e.g., Laponite®), a hydrophilic polymer (e.g., poly(ethylene oxide) (PEO)), a thickening polymer (e.g., CMC), a plurality of oxidized metal particles (e.g., zinc oxide particles), or a polymer comprising a plurality of quaternary ammonium functional groups (e.g., chitosan functionalized with quaternary ammonium functional groups).

The composition of the invention may comprise or consist of water, a plurality of silicate nanoparticles (e.g., Laponite®), a hydrophilic polymer (e.g., poly(ethylene oxide) (PEO)), a thickening polymer (e.g., CMC), a plurality of oxidized metal particles (e.g., zinc oxide particles), and a polymer comprising a plurality of quaternary ammonium functional groups (e.g., chitosan functionalized with quaternary ammonium functional groups).

The composition of the invention may comprise or consist of water, glycerol, a plurality of silicate nanoparticles (e.g., Laponite®), a hydrophilic polymer (e.g., poly(ethylene oxide) (PEO)), a thickening polymer (e.g., CMC), a plurality of oxidized metal particles (e.g., zinc oxide particles), an adhesive polymer (e.g. poly(vinyl acetate)), and a polymer comprising a plurality of quaternary ammonium functional groups (e.g., chitosan functionalized with quaternary ammonium functional groups).

The composition of the invention may comprise or consist of water, glycerol, a plurality of silicate nanoparticles (e.g., Laponite®), a hydrophilic polymer (e.g., poly(ethylene oxide) (PEO)), a thickening polymer (e.g., CMC), a plurality of oxidized metal particles (e.g., zinc oxide particles), an adhesive polymer (e.g. poly(vinyl acetate)), or a polymer comprising a plurality of quaternary ammonium functional groups (e.g., chitosan functionalized with quaternary ammonium functional groups).

The composition of the invention may comprise or consist of water, a plurality of silicate nanoparticles (e.g., Laponite®) and a hydrophilic polymer (e.g., poly(ethylene oxide) (PEO)).

The composition of the invention may comprise or consist of water, a plurality of silicate nanoparticles (e.g., Laponite®), a hydrophilic polymer (e.g., poly(ethylene oxide) (PEO)) and an adhesive polymer (e.g. poly(vinyl acetate)).

The composition of the invention may comprise or consist of water, a plurality of silicate nanoparticles (e.g., Laponite®), a hydrophilic polymer (e.g., poly(ethylene oxide) (PEO)), an adhesive polymer (e.g. poly(vinyl acetate)) and a plurality of oxidized metal particles (e.g., zinc oxide particles).

The composition of the invention may comprise or consist of water, a plurality of silicate nanoparticles (e.g., Laponite®), a hydrophilic polymer (e.g., poly(ethylene oxide) (PEO)), an adhesive polymer (e.g. poly(vinyl acetate)) and a polymer comprising a plurality of quaternary ammonium functional groups (e.g., chitosan functionalized with quaternary ammonium functional groups).

The composition of the invention may comprise or consist of water, a plurality of silicate nanoparticles (e.g., Laponite®), a hydrophilic polymer (e.g., poly(ethylene oxide) (PEO)), an adhesive polymer (e.g. poly(vinyl acetate)) a plurality of oxidized metal particles (e.g., zinc oxide particles) and a polymer comprising a plurality of quaternary ammonium functional groups (e.g., chitosan functionalized with quaternary ammonium functional groups).

The composition of the invention may comprise or consist of water, glycerol, a plurality of silicate nanoparticles (e.g., Laponite®) and a hydrophilic polymer (e.g., poly(ethylene oxide) (PEO)).

The composition of the invention may comprise or consist of water, glycerol, a plurality of silicate nanoparticles (e.g., Laponite®), a hydrophilic polymer (e.g., poly(ethylene oxide) (PEO)) and an adhesive polymer (e.g. poly(vinyl acetate)).

The composition of the invention may comprise or consist of water, glycerol, a plurality of silicate nanoparticles (e.g., Laponite®), a hydrophilic polymer (e.g., poly(ethylene oxide) (PEO)), an adhesive polymer (e.g. poly(vinyl acetate)) and a plurality of oxidized metal particles (e.g., zinc oxide particles).

The composition of the invention may comprise or consist of water, glycerol, a plurality of silicate nanoparticles (e.g., Laponite®), a hydrophilic polymer (e.g., poly(ethylene oxide) (PEO)), an adhesive polymer (e.g. poly(vinyl acetate)) and a polymer comprising a plurality of quaternary ammonium functional groups (e.g., chitosan functionalized with quaternary ammonium functional groups).

The composition of the invention may comprise or consist of water, glycerol, a plurality of silicate nanoparticles (e.g., Laponite®), a hydrophilic polymer (e.g., poly(ethylene oxide) (PEO)), an adhesive polymer (e.g. poly(vinyl acetate)) a plurality of oxidized metal particles (e.g., zinc oxide particles) and a polymer comprising a plurality of quaternary ammonium functional groups (e.g., chitosan functionalized with quaternary ammonium functional groups).

The composition as a gel sealant may comprise a combination of aqueous media and an organic liquid (such as glycerol).

The composition of the invention may comprise or consist of

1 to 15 wt %, or 8 to 15 wt % plurality of silicate nanoparticles;

0.5 to 10 wt %, or 2 to 6 wt % hydrophilic polymer;

up to 100 wt % water; and optionally further comprising

0 to 9 wt % thickening agent;

0.2 to 5 wt %, or 0.2 to 0.6 wt % plurality of zinc oxide particles;

0.2 to 2 wt %, or 0.3 to 0.5 wt % polymer comprising a plurality of quaternary ammonium functional groups;

0.1 to 50 wt %, or 0.5 to 2 wt % adhesive polymer; and

20 to 80 wt % or 35 to 55 wt % glycerol.

The composition of the invention may comprise or consist of

1 to 15 wt %, or 8 to 15 wt % plurality of silicate nanoparticles;

0.5 to 10 wt %, or 2 to 6 wt % hydrophilic polymer;

0.1 to 50 wt %, or 0.5 to 2 wt % adhesive polymer;

20 to 80 wt % or 35 to 55 wt % glycerol, and

up to 100 wt % water; and optionally further comprising

0.2 to 5 wt %, or 0.2 to 0.6 wt % plurality of zinc oxide particles;

0.2 to 2 wt %, or 0.3 to 0.5 wt % polymer comprising a plurality of quaternary ammonium functional groups;

The composition of the invention may comprise or consist of

1 to 15 wt %, or 8 to 15 wt % plurality of lithium magnesium sodium silicate nanoparticles;

0.5 to 10 wt %, or 2 to 6 wt % poly(ethylene oxide) having a molecular weight from about 1 kDa to about 10,000 kDa;

0.1 to 50 wt %, or 0.5 to 2 wt % polyvinylacetate;

20 to 70 wt % or 35 to 55 wt % glycerol, and

up to 100 wt % water; and optionally further comprising

0.2 to 5 wt %, or 0.2 to 0.6 wt % plurality of zinc oxide particles and/or

0.2 to 2 wt %, or 0.3 to 0.5 wt % chitosan comprising a plurality of quaternary ammonium functional groups.

The composition as defined herein may further comprise one or more drug or agent.

The one or more drug may be selected from the group comprising antibiotic drug, antiparasitic drugs, antimycotic drugs, analgesics or anti-inflammatory drugs, vitamins, corticosteroid drugs and proteins. The one or more agent may b selected from the group comprising coloring agents or dyes and colorimetric pH indicators.

The composition may further comprise one or more antibiotic drugs. The antibiotic drug may be selected from the group comprising or consisting of penicillins such as penicillin, penicillin G, hetacillin potassium, cloxacillin benzathine, ampicillin and amoxicillin trihydrate, aminocoumarins such as novobiocin, cephalosporins such as cephalexin, ceftiofur sodium, ceftiofur hydrochloride, ceftiofur crystalline free acid, macrolides such as tildipirosin, tylosin, tulathromycin, erythromycin, clarithromycin, and azithromycin, quinolones and fluoroquinolones such as enrofloxacin, ciprofloxacin, levofloxacin, and ofloxacin, sulfonamides such as sulfadimethoxine, co-trimoxazole and trimethoprim, tetracyclines such as tetracycline, oxytetracycline and doxycycline, aminoglycosides such as dihydrostreptomycin sulfate, neomycin, gentamicin and tobramycin, lincosamides such as pirlimycin hydrochloride, lincomycin, clindamycin, and pirlimycin, and amphenicols such as florfenicol.

The composition may further comprise one or more antiparasitic drugs. The antiparasitic drug may be selected from the group comprising or consisting of antiprotozoals such as melarsoprol, eflornithine, metronidazole, tinidazole, miltefosine, antihelminthics such as mebendazole, pyrantel pamoate, thiabendazole, diethylcarbamazine, ivermectin, aticestodes such as niclosamide, praziquantel, albendazole, antitrematodes such as praziquantel, antiamoebics such as rifampin and amphotericin B, and broad-spectrum drugs such as nitazoxanide.

The composition may further comprise one or more antimycotic drugs. The antimycotic drug may be selected from the group comprising or consisting of polyenes such as amphotericin b, candicidin, filipin, hamycin, natamycin, nystatin, and rimocidin; azoles such as imidazole, triazole, thiazole, bifonazole, butoconazole, clotrimazole, econazole, fenticonazole, isoconazole, ketoconazole, luliconazole, miconazole, omoconazole, oxiconazole, sertaconazole, sulconazole, tioconazole, albaconazole, efinaconazole, epoxiconazole, fluconazole, isavuconazole, itraconazole, posaconazole, propiconazole, ravuconazole, terconazole, voriconazole, and abafungin; allylamines such as amorolfin, butenafine, naftifine, and terbinafine; echinocandins such as anidulafungin, caspofungin and micafungin; and others such as aurones, benzoic acid, ciclopirox olamine, flucytosine or 5-fluorocytosine, griseofulvin, haloprogin, tolnaftate, undecylenic acid, triacetin, crystal violet, castellani's paint, orotomide (f901318), miltefosine, potassium iodide, coal tar, copper(ii) sulfate, selenium disulfide, sodium thiosulfate, piroctone olamine, iodoquinol, clioquinol, acrisorcin, zinc pyrithione, and sulfur.

The composition may further comprise one or more analgesics or anti-inflammatory drugs. The analgesic or anti-inflammatory drug may be selected from the group comprising or consisting of aspirin, ibuprofen, and naproxen, naproxen sodium, diclofenac, acetoaminophen, celecoxib, piroxicam, indomethacin, meloxicam, ketiprofen, sulindac, diflunisal, nabumetone, oxaprozin, tolmetin, salsalate, etodolac, fenoprofen, flurbiprofen, ketorolac, meclofenamate, and mefenamic acid.

The composition may further comprise one or more corticosteroid drugs. The corticosteroid drug may be selected from the group comprising or consisting of prednisone, betamethasone, cortisone, dexamethasone, hydrocortisone, methylprednisolone, prednisolone and triamcinolone acetonide.

The composition may further comprise one or more proteins. The protein may be selected from the group comprising or consisting of an enzyme, an hormone (progesterone, estrogens), an antibody, a CAS protein, a transmembrane protein, an amino acid, a cell signaling proteins, and a structural protein such as collagen, hyaluronan, elastin, and tropoelastin.

The composition may further comprise one or more coloring agents or dyes. The coloring agent or dye may be selected from the group comprising or consisting of Quinoline yellow, Ponceau 4R, Carmoisine, Patent Blue V, Greens S, Brilliant Blue FCF, lndigotine, Fast Green FCF, Erythrosine, Sunset Yellow, Allura Red AC, Tartrazine, Sunset Yellow FCF, Spirulina, and Betanin.

The composition may further comprise one or more colorimetric pH indicators. The colorimetric pH indicator may be selected from the group comprising or consisting of bromocresol purple, bromothymol blue, methyl red, and phenol red.

The composition is in the form a gel. The gel may have a density from 0.5 g/ml to 3.0 g/ml or from 0.8 g/ml to 2.0 g/ml or from 1 g/ml to 1.5 g/ml at about 38° C.

The sol-gel polymer composition has shear thinning properties such that the composition can be deformed in a syringe at room temperature. The composition is capable of being injected using a single-barrel syringe and the like. The composition gels immediately upon injection (for example, in less than 60 seconds, or in less than 30 second, or in less than 10 seconds).

The composition has viscoelastic and recoverability properties. For example, when 100% strain is applied to the gel (outside the viscoelastic region of the gel) for 5 min, the gel deforms. After removal of 100% strain, 1% strain is applied for 5 min, and the gel quickly recovers to its original structure, thus suggesting no permanent deformation of the hydrogel. The gel is injectable with injection force <100 N with tip a diameter of about 1.5 mm at an administration rate of about 2.5 ml/s.

the composition has mechanical strength from about 10² Pa to about 10⁵ Pa at strain value from about 10⁻¹% to about 10³%.

The composition is shear-thinning. Spreadability may be measured, for example, by the procedures described in Lardy, F., et al. Functionalization of hydrocolloids: Principal component analysis applied to the study of correlations between parameters describing the consistency of hydrogels. Drug development and industrial pharmacy 26, 715-721 (2000) or Garg, A., et al. Spreading of semisolid formulations: an update. Pharmaceutical Technology North America 26, 84-84 (2002).

The composition is mucoadhesive (for example, due to the presence of the chitosan component). Mucoadhesive properties may be measured, for example, by the procedures described in Shaikh, R., et al. Mucoadhesive drug delivery systems. J Pharm Bioallied Sci 3, 89-100, doi:10.4103/0975-7406.76478 (2011), Annabi, N. et al. Engineering a highly elastic human protein-based sealant for surgical applications. Sci Transl Med 9, eaai7466, doi:10.1126/scitranslmed.aai7466 (2017), or Example 12.

The composition is stable at about 23° C. for up to 120 days or the composition is stable at about 37° C. for up to 120 days, the composition is stable at about 39° C. for up to 120 days. The composition is stable in the presence of milk or water at 37-40° C. for up to about 120 days.

Due to their shear-thinning properties, application of the compositions is reversible; in other words, the gels may be removed from their application site by applying shear stress (squeezing or pressure).

When combined with the other components of the composition, a physically reversible gel is formed. If certain ions are present in the composition, they may electrostatically shield the interactions, thereby impairing the nanoplatelets from assembling. For example, pyrophosphates absorb onto the positively charged edge, thereby impairing formation of the three-dimensional structure. This strategy can be employed to reverse gelation; the gel may be easily removed upon addition of a salt. The composition may be removed by the addition of saline solutions that optionally comprise tetrasodium pyrophosphate or low molecular weight glycols. The saline solution may comprise 1-15 wt % tetrasodium pyrophosphate. The low molecular weight glycol may be ethylene glycol or polyethylene glycol with a molecular weight of 50 to 1000 g/mol.

Medical Use

The composition as defined herein or gel may be used to treat topical injuries and prevent infection by serving as a physical microbial barrier. The gel may be used as an intra-mammary teat sealant to treat or prevent mastitis in dairy cattle. The gel may be administered or applied easily, for instance by injecting with a syringe.

The composition is antimicrobial or bacteriostatic. The composition exhibits a MIC₅₀ of at least 40 μg/mL against S. aureus or E. coli, or both.

The invention relates to a method of treating or preventing mastitis, comprising applying in an intramammary teat canal of a subject in need thereof an effective amount of any of the compositions described herein, thereby treating or preventing mastitis.

The subject may be a dairy livestock animal, preferably a heifer or a cow, but also can include other animals such as goats, sheep, horses, water buffaloes and the like. The invention relates to any of the methods described herein, wherein the subject is a cow.

The sol-gel polymer composition may be administering comprises applying topically the composition on mammary tissue or applying within the teat canal the sol-gel polymer composition. Topical administration may comprise dipping the teat in the composition. The composition may be administered via intramammary administration, for example, by injection or infusion.

The composition may be administered when the animal begins to dry off or during the dry period of the animal. The composition remains in the teat canal during the dry off period, thus reducing the clinical and sub-clinical cases of mastitis during the dry off period and in the first stage (post calving) of lactation. By remaining in the teat canal throughout the dry period, the composition minimizes microbial invasion through the teat canal during high risk periods in the pre-fresh dairy animal. The composition may be administered prior to infection of a healthy animal. The composition may be administered during the postpartum period of a non-lactating animal. The composition may be administered during the prepartum period of an animal. The milk obtained from the animal after application may be used in the production of a milk product.

The invention relates to any of the methods described herein, further comprising removing the composition from the intramammary teat canal. The invention relates to any of the methods described herein, wherein the composition is removed by applying shear stress to the teat. The invention relates to any of the methods described herein, wherein the composition is removed by contacting the composition with a saline solution.

The invention relates to a method of treating or preventing a topical, subcutaneous, or internal infection in a subject in need thereof, comprising locally administering to the subject an effective amount of any of the compositions described herein, thereby treating or preventing the infection.

The invention relates to any of the methods described herein, wherein the infection is a skin ulcer, a diabetic ulcer, acne, rosacea, an abscess, or an organ infection.

The invention relates to a method of treating a skin wound, such as a laceration, an abrasion, a puncture, an avulsions, an ulcer, and a burn in an animal and or human subject in need thereof, comprising locally administering to the subject an effective amount of any of the compositions described herein, thereby treating the skin wound.

The composition, upon administration, forms a physical barrier, thereby substantially impeding cell infiltration to the gel.

The composition provides medical and veterinary uses for the sol-gel polymer compositions. While human applications will become apparent from the disclosure, a preferred use relates to a method of forming a physical barrier in the teat canal of a dairy animal for the treatment or prevention of mammary diseases that may occur as the animal begins to dry off or during the dry period, comprising administering to the teat or within the teat canal of the animal any of the sol-gel polymer compositions described herein. The composition may be used in methods of preventing infection or mastitis by preventing the invasion of the mammary gland by mastitis-causing microorganisms. The composition may be used for the treatment or prevention of microbial mastitis or for the treatment or prevention of the microbial infection of topical injuries, treatment of skin wounds such as lacerations, abrasions, punctures, avulsions, ulcers and burns in an animal and or human subject through locally administering the composition.

The invention relates to a method of delaying or preventing pregnancy in a subject comprising administering to the fallopian tubes of the subject any of the compositions described herein in an amount sufficient to substantially impede or prevent fluid communication between the ovary and the uterus.

The invention relates to a kit comprising the composition as defined herein one or more syringes, together with instructions for use and optionally further including the saline solutions as defined above for removal of the composition after administration.

### EXAMPLES

In order that aspects of the invention described herein may be more fully understood, the following examples are set forth. The examples described in this application are offered to illustrate the compounds, pharmaceutical compositions, and methods provided herein and are not to be construed in any way as limiting their scope.

Materials and Methods

Chemicals and solvents were purchased from commercial suppliers or were fabricated and purified by standard techniques. The following commercial reagents were used as purchased without any further purification: Chitosan (Low molecular weight, Sigma Aldrich 448869), glycidyltrimethylammonium chloride aqueous solution (containing 20-25% water, Sigma Aldrich 50053). poly(ethylene glycol) (Mn=20 kDa, Sigma-Aldrich 81300), poly(ethylene glycol) (Mn about 8, 000,000 kDa, Sigma-Aldrich 372838), deuterium oxide (Sigma-Aldrich 1133660025).

### Example 1—Synthesis of Quaternary Chitosan

Quaternary chitosan (qCh) was prepared as previously described with minor modifications (Viviane A. et al. Preparation and characterization of quaternary chitosan salt: adsorption equilibrium of chromium (VI) ion. Reactive & Functional Polymers 61 (2004) 347/352). Chitosan (50,000-190,000 Da (based on viscosity); 75-85% deacetylated) (5.0 g) and 100 mL of deionized (DI) water were added into a 250 mL round-bottomed flask. Subsequently, the mixture was stirred at room temperature (about 23° C.) for 30 min (the chitosan did not completely dissolve). Afterwards a glycidyltrimethylammonium chloride aqueous solution (≥90%) (5.0 g) was added. The reaction flask was closed by a sleeve stopper. Next, a house vacuum was employed to evacuate the system through a needle sticking in the stopper. Then the reaction mixture was heated to 100° C. using a hot plate and an oil bath. After 15 s when the mixture started to boil, the needle connected to the vacuum was removed. This procedure was performed in order to create an inert atmosphere to remove the oxygen in the reaction flask. The mixture was stirred at the stated temperature for 24 h. Then, the mixture was cooled to room temperature and added, in equal proportions, to 12×50 ml centrifuge tubes containing 30 ml ethanol each. After centrifugation, the supernatant was discarded and the precipitate rinsed three times with ethanol. Next, the product was stored in a vacufuge to dry it by using a “D-AQ” mode overnight with the temperature set at 30° C. The reaction was characterized using ¹H-NMR (Nuclear magnetic resonance) spectroscopy by dissolving the dry product in D₂O. Data were analyzed using specialized software compatible with Bruker 600 NMR. The degrees of quaternization and deacetylation were calculated from the integration area of the peaks at around 3.23 ppm (—N(CH₃)₃⁺), 2.81 ppm (1H at the pyranose ring), and 2.06 ppm (—C(O)CH₃).

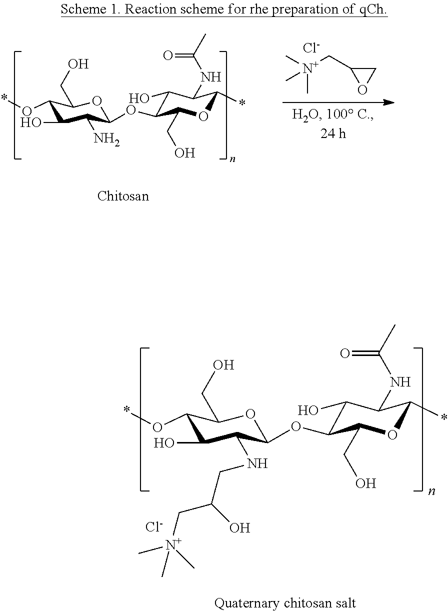

### Example 2—Representative Example of Gel Fabrication

A shear-thinning antibacterial sealant hydrogel was formulated by mixing several components. Initially, Laponite® (10 g, Laponite-XLG XR, BYK) was added to 80 ml of cold water (4° C.) and mixed by a laboratory homogenizer at 800 rpm at room temperature for 15 min or until the gel was fully formed. In a separate beaker, PEO (Mw=20 kDa, Sigma Aldrich 8.21037) (3 g, 15 wt. %), qCh (1 g, 5 wt. %), and varying amounts of carboxymethyl cellulose (CMC sodium salt, viscosity=500-900 mPa·s at 2% in water at 25° C., TCI America C0045) (2 g-4 g, 10-20 wt. %) were homogenized in DI water (20 ml) at 80° C. by stirring it for 30 min to form a stock solution. The stock solution (20 ml) was then added to the Laponite® gel (80 ml) and vigorously mixed by a homogenizer at about 1,200 rpm. The resulting hydrogel was further stabilized by incubating it at room temperature for 12 h before use.

### Example 3—ZnO-Containing Gel Fabrication

A shear-thinning antibacterial sealant hydrogel was formulated by mixing several components. Initially, PEO (Mw=20 kDa, Sigma Aldrich 8.21037) (3 g) was dissolved in 80 ml of Milli-Q water. This solution was cooled to 4° C., and Laponite (10 g, Laponite-XLG XR, BYK) and ZnO nanoparticles (ZnO NPs, particle size <100 nm (TEM), 40 nm average particle size, 20 wt % in H₂O, Sigma Aldrich 721077) (1 g) were added, followed by mixing with a laboratory homogenizer at 800 rpm at room temperature for 15 min or until the gel was fully formed. In a separate beaker, qCh (1 g, 5 wt. %), and varying amounts of carboxymethyl cellulose (CMC sodium salt, viscosity=500-900 mPa·s at 2% in water at 25° C., TCI America C0045) (2 g-4 g, 10-20 wt. %) were homogenized in DI water (20 ml) at 80° C. by stirring it for 30 min to form a stock solution. The stock solution (20 ml) was then added to the Laponite gel (80 ml) and vigorously mixed by a homogenizer at about 1,200 rpm. The resulting hydrogel was further stabilized by incubating it at room temperature for 12 h before use.

### Example 4—Fabrication of Binary Gel with Glycerin and ZnO

The binary gel with anti-freezing, anti-drying and antibacterial properties was formulated by the addition of glycerin. PEO (Mw ˜20 kDa, Sigma Aldrich 8.21037) (4 g) and 0.4 g of PEO (Mw˜8,000 kDa) were dispersed in 44 g of glycerin (Sigma Aldrich 1.37028). This mixture was heated at 90° C. by water bath for 5 min. Next, 34 ml of Milli-Q water were added, followed by mixing until the polymer dissolved completely. ZnO NPs (2.5 g, Sigma Aldrich 721077) were added and the mixture was further mixed until its temperature reached to room temperature (about 23° C.). For gel formulations containing the pH indicator, 80 μL of bromocresol purple (10 mg/ml in ethanol, Sigma Aldrich 114375) could be optionally added in this step. Fifteen grams of Laponite were then added, mixed with the solution prepared above until a gel was formed, and incubated at 90° C. by water bath for 20 min to stabilize the gel. After the gel was cooled down to 50° C. or lower, 7.167 g qCh solution (7.5% w/w in water), 11 g of glycerin and 2.333 g of Milli-Q water were added and mixed vigorously. The gel was further mixed well with 12.5 ml of 0.1 g/ml poly (vinyl acetate) (Mw˜100 kDa, Sigma Aldrich 189480) in ethyl acetate (HPLC Plus, Sigma Aldrich 650528) and 300 μL of acetic acid glacial (HPLC, Sigma Aldrich AX0074). Finally, the gel mixture was processed at 60° C. under vacuum for 5 h.

### Example 5—Evaluation of Gel Antimicrobial Properties

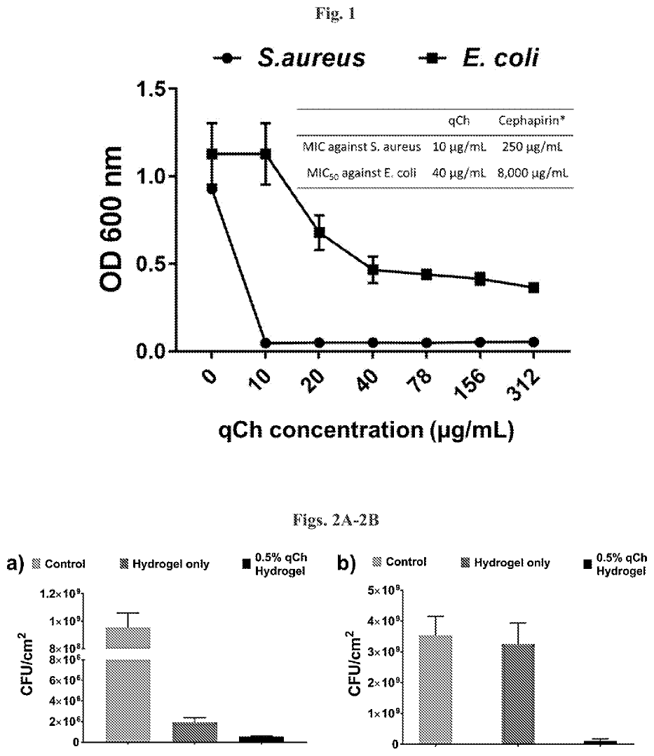

Staphylococcus aureus (S. aureus) and Escherichia coli (E. coli) strains used in all the experiments were isolated from dairy farms. Prior to each experiment, bacterial stock cultures were prepared by inoculating a single bacterial colony in 3.75% Brain Heart Infusion (BHI, Sigma Aldrich 53286) broth at 37° C. until mid-log phase. Bacterial stock cultures were then diluted to 10⁹ colony forming unit (CFU)/ml. To test the antibacterial properties of quaternary chitosan (qCh) in a solution, different concentrations of the qCh from 10 to 312 μg/ml were serially diluted in BHI broth. Bacteria solution was then added to the freshly prepared qCh solutions on a 96-well plate. The final bacterial seeding density was 5*10⁵ CFU/ml. The control sample contained bacteria growing in BHI broth without qCh. After 24 hours of incubation, bacterial density was measured by optical density (OD) at a wavelength of 600 nm (OD 600 nm). See FIG. 1.

The antibacterial properties of the gels were compared to Orbeseal® Dry Cow Teat Sealant (bismuth subnitrate (65% w/w) in an oil base). Briefly, 0.2 ml of the antimicrobial gel of Example 2 and 0.2 ml of Orbeseal were added (in separate wells) to a 96-well plate (1 ml deep well). Samples were then centrifuged and 20 μL of bacterial solution (10⁵ CFU/ml) was added to each sample. The control wells consisted of BHI broth containing 10⁵ CFU/ml of the mentioned bacterial strains. Samples were incubated at 37° C. for predetermined time points. After incubation, samples were resuspended in autoclaved water, serially diluted in phosphate buffered saline (PBS, pH 7.4), and plated on sheep blood agar plates. The agar plates were then incubated for another 16 hours. Bacterial density on each sample (CFU/cm²) was determined by the number of colonies counted. See FIGS. 2A-2B, 3A-3B, and 4A-4B.

### Example 6—Injection Force Determination

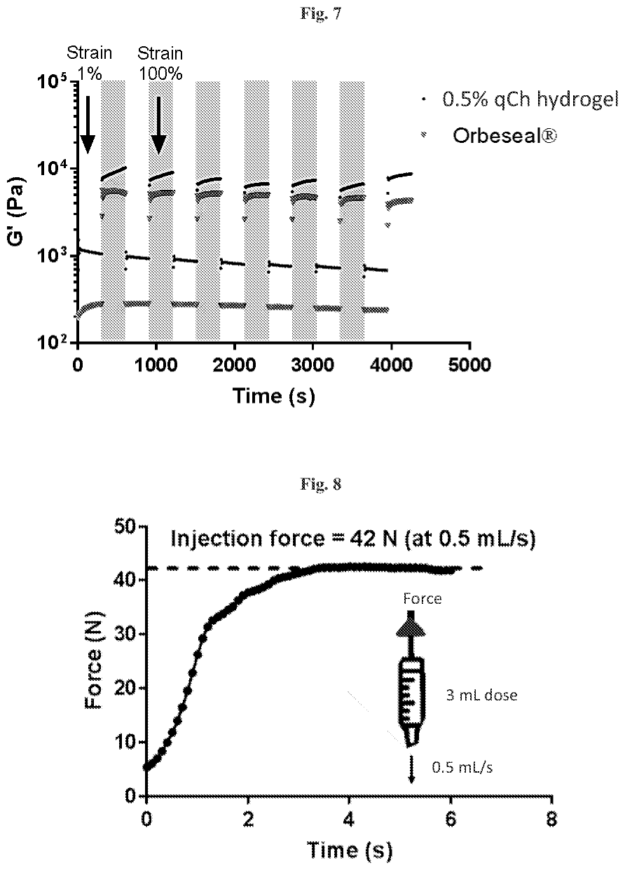

The injectability of the materials was analyzed using a mechanical tester (Instron Model 5943, Norwood, Mass.) equipped with a 500 N load cell. Briefly, the gel was added to a 5 ml Luer lock syringe with a flow rate of 0.5 ml/s. The syringe without a needle was plugged in a custom-made holder. The syringe plunger was depressed using an upper compressive platen. The force was recorded using Bluehill version 3 software (Instron, Norwood, Mass.). All the measurements were repeated at least three times. The holder was cleaned after each repetition. See FIG. 8.

### Example 7—Evaluation of Rheological Properties

The rheology tests were performed on a TA Instruments DHR-2 Rheometer at 37° C. using a 40 mm cone. A fresh new sample was employed for every test or repetition. The temperature soak time was 20 s before the first measurement of every sample. The oscillation frequency was 1 Hz. Two methods were performed as followed:

Strain Sweep

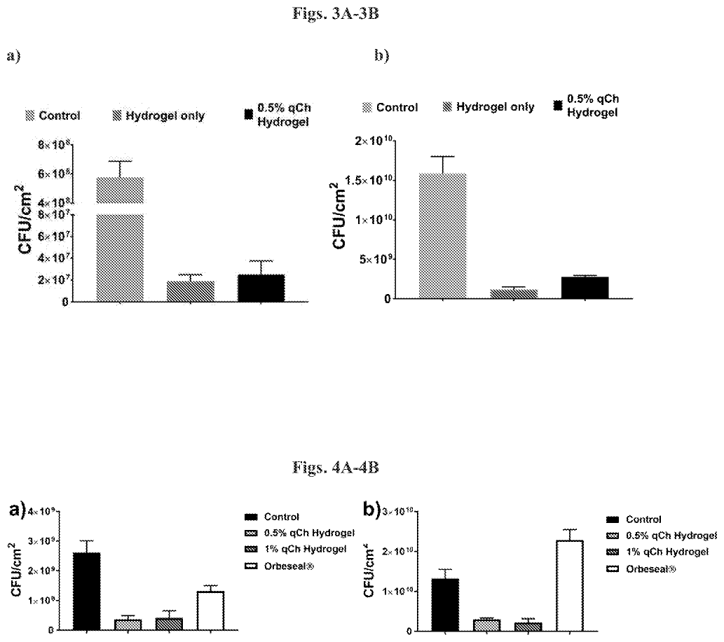

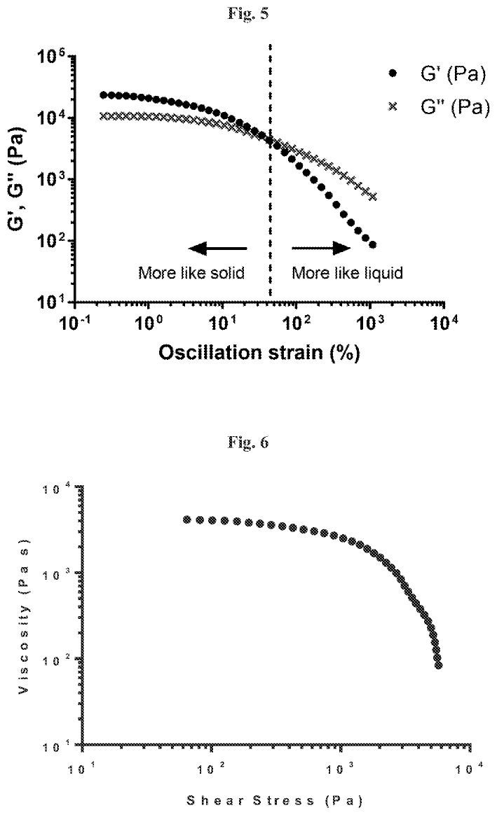

The strain was increased logarithmically from 0.01% to 1000%. 10 data points were acquired per decade. The yield stress and yield strain were identified by modulus crossover in the G′, G″ vs. stress and G′, G″ vs. strain plots respectively. See FIG. 5. The viscosity vs. stress plot was also reported. See FIG. 6. The strain sweep experiment was repeated at least three times for every material.

Recovery Test

High-strain oscillation was performed at 100% strain (higher than the yield strain) for 300 s, which was followed by low-strain (1%) oscillation for another 300 s. This procedure was repeated 7 times continuously. G′ vs. time plot was reported, which demonstrated the recoverability of the mechanical properties of the samples after forcing them to yield. See FIG. 7.

### Example 8—Density and pH Determination

Density measurements were performed by dividing the mass of the gel by its volume at room temperature

The pH of the gels was investigated by dissolving the 1 ml of each gel sample completely in 10 ml of MilliQ water, followed by measuring the pH of the gel suspension with a pH meter. See Table. 1

Table 1. Density and pH values of Laponite gel (hydrogel only), 0.5% qCh gel (formulated as described in Example 2), and 1% ZnO NPs/0.5% ZnO hydrogel (formulated as described in Example 3).

| Gel sample | Density (g/ml) | pH |
|---|---|---|
| Hydrogel only | 1.10 | 9.46 |
| (3% PEO/8% CMC/10% Laponite) | | |
| 0.5% qCh hydrogel | 1.01 | 9.54 |
| (0.5% qCh/3% PEO/8% CMC/10% Laponite) | | |
| 1% ZnO NPs/0.5% qCh hydrogel | 1.06 | 10.54 |
| (1% ZnO NPs/0.5% qCh/ 3% PEO/8% CMC/10% | | |
| Laponite) | | |
| Binary gel with glycerol | 1.08 | 10.03 |
| (0.5% qCh/0.5% ZnO/3% PEO 20k/l% PEO | | |
| 300k/10% Laponite; solvent: glycerol-water | | |
| (2:1, w/w) mixture) | | |

### Example 9—Gel Stability

The gel stability was tested in physiological buffer conditions in vitro. 1 g of each gel sample (hydrogel only or 0.5% qCh hydrogel) was placed onto a cell container with a 100 μm filter pore size (Falcon, N.C., USA). The weights of empty cell containers were also measured prior adding the gels. Samples were placed in either PBS (DPBS with calcium and magnesium, pH 7.4, Fisher 14080055) or PBS with 15 μg/ml lysozyme (from chicken egg white, Sigma Aldrich, USA) and incubated at 37° C. over 8 weeks. At each pre-determined time interval, each container with gel was weighed after carefully removing excessive liquid on the bottom. The net weight of the gel was then calculated by subtracting the weight of the empty container. Each experimental group was triplicated. See FIGS. 9A and 9B.

### Example 10—Thermal Analysis

The experiment was conducted using a TA Instruments DSC Q100 differential scanning calorimeter. The temperature decreased from room temperature to −50° C. at a rate of 5° C./min, and then increased from −50° C. to 100° C. also at a rate of 5° C./min. The baseline was measured by having two empty pans. Then, one of the empty pans was loaded with 9.5 mg of gel, and the other was untouched and used as the reference. The results were reported with the baseline subtracted.

### Example 11—Spreadability

The spreadability test was performed at room temperature 48 h after the preparation of the gels by the parallel plate method. The gel was loaded in the central hole of a glass plate mold. The hole has a diameter of 1 cm. Specifically, 1.0 g±0.01 g of the gel was placed between two horizontal glass plates (20×20 cm), and the gel was pressed by plates of known weights. Subsequently, after 1 minute the spreading diameter was measured and classified according to the spreading diameter as following:
Fluid gel Ø>70 mm
Semifluid gel 70≥Ø>55 mm
Semistiff gel 55≥Ø>47 mm
Stiff gel 47≥Ø>40 mm
Very stiff gel Ø≤40 mm

### Example 12—Tissue/Mucoadhesive Property Tests Through Lap Shear Experiments

The lap shear experiments where performed according to the modified ASTM F2255-05 standard for lap shear strength property of tissue adhesives. Fresh porcine skin was obtained from a local slaughterhouse and they were immersed in PBS before use to prevent drying. Two pieces of fresh porcine skin were cut with 4 cm length, 1 cm width and 2 mm thickness. These two pieces were glued separately using super glue to glass slides (7.5 cm×2.5 cm). To prepare the test 80 μL of the gel were applied in a 1 cm² area of one of the tissues and the other piece was immediately paced over the gel and force applied by using clamp for 5 min at 37° C. in wet plastic bag prior measurements. The samples were then immediately strained until failure at a cross-head speed of 10 mm/min. For each material at least three repetitions were performed.

### Example 13—Gel Composition

Ingredients:

#1: No ZnO, no qCh

#2: ZnO only (0.5 g), no qCh

#3: qCh only (0.5 g), no ZnO

#4: Both ZnO (0.5 g) and qCh (0.5 g)

Other ingredients added: Glycerol (55 g), water (45 g, but part of it evaporates in the vacuum drying process), Laponite (15 g), PEO 20 k (4 g), poly(vinyl acetate) (1.25 g), PEO 300 k (0.4 g).

Gel Making Protocol:

10 g of poly(vinyl acetate) was dissolved in ethyl acetate to make a 100 ml solution for later use. 4.00 g of PEO (20 k molecular weight), 0.400 g of PEO (300 k molecular weight), 55 g (44 g for gel #3 and #4) of glycerol, and 45 g (43 g for gel #2, and 34 g for gel #4) of water were added to a 600 ml glass beaker. A homogenizer equipped with a PTFE-coated cross impeller was used for stirring. The beaker was put on a hot plate with a thermocouple dipping into the solution for temperature control. The beaker was wrapped with multiple pieces of food wrap to prevent water evaporation. The mixture was stirred at 1250 rpm throughout a 43 min PEO dissolving process. It was first heated up to 80° C. (took 13 min), then let it cool down to and stay at 40° C., which took another 30 min. For gel #2 and #4 only, 2.50 g of 20% ZnO nanoparticle aqueous dispersion was added and stirred for 1 min. 15 g of Laponite was added and it formed a gel within about 1 min. The temperature was kept at 40° C. and the stir rate was kept at 1250 rpm until the gel formation. The stirring and heating were stopped immediately once the gel was formed. For gel #3 and #4 only, 9.5 g of the qCh aqueous solution (contains 0.5 g of qCh and 9 g of water) and 11 g glycerol were added, then stirred and mixed with the gel at 320 rpm for 10 min. 12.5 ml of the 10% poly(vinyl acetate) solution and 70 uL (300 uL for #3 and #4 gel) of acetic acid glacial were added, then stirred at 320 rpm for 7 min before stirring at 2500 rpm for another 10 min. The gel looked very adhesive as it sticks to the beaker at this point. The beaker was wrapped by a piece of food wrap with several small holes poked. It was then placed in a vacuum oven (Eppendorf Vacufuge plus) to evaporate the ethyl acetate and some of the water to strengthen the gel. The setting was 60° C. and “D-AQ.” After 75 min of evaporation, it was taken out for 1 min of 2500 rpm stirring, then continued to evaporate for another 75 min. Finally, it was stirred at 2500 rpm for 5 min.

### Example 14—Experiment for the Clinical Trial of Burn Wound Healing in Porince Model

1. Wound creation of 3 cm×3 cm 200 C metal block 10 seconds or 20 seconds contact.

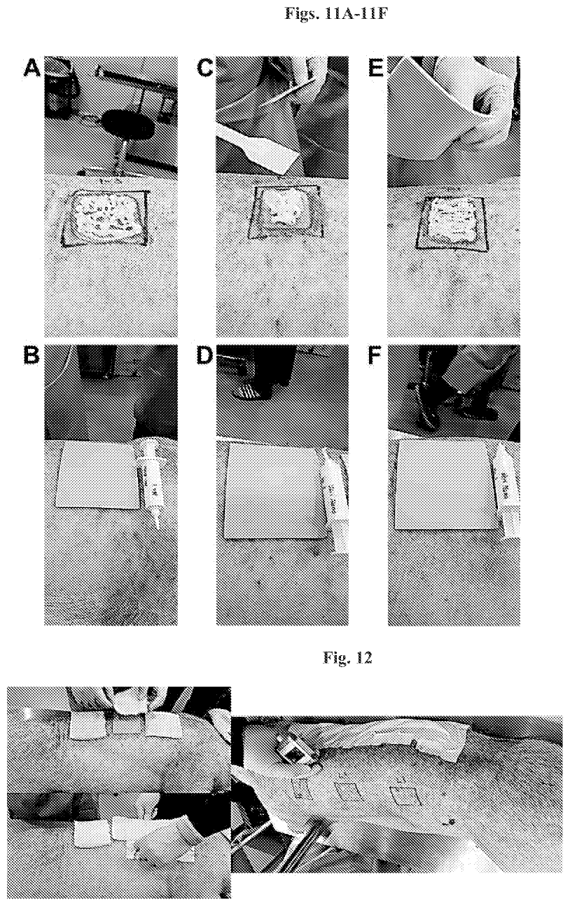

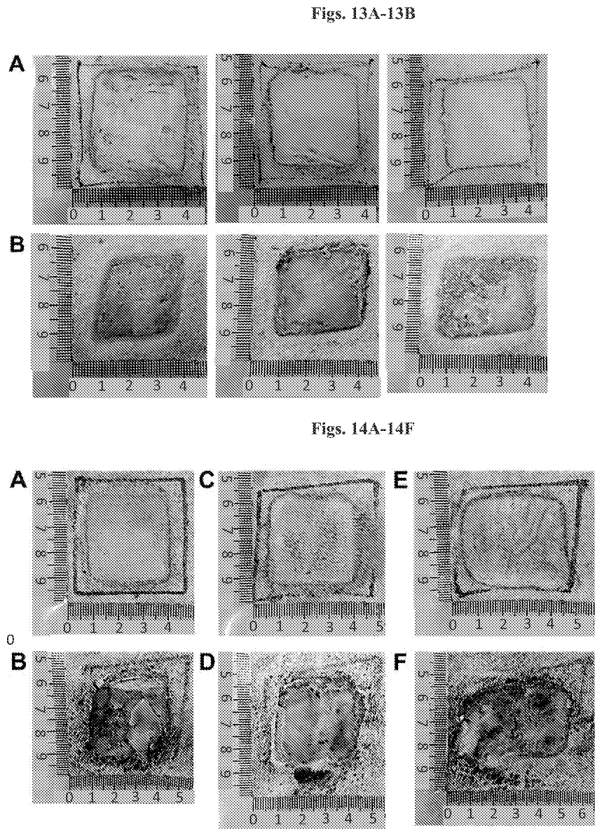

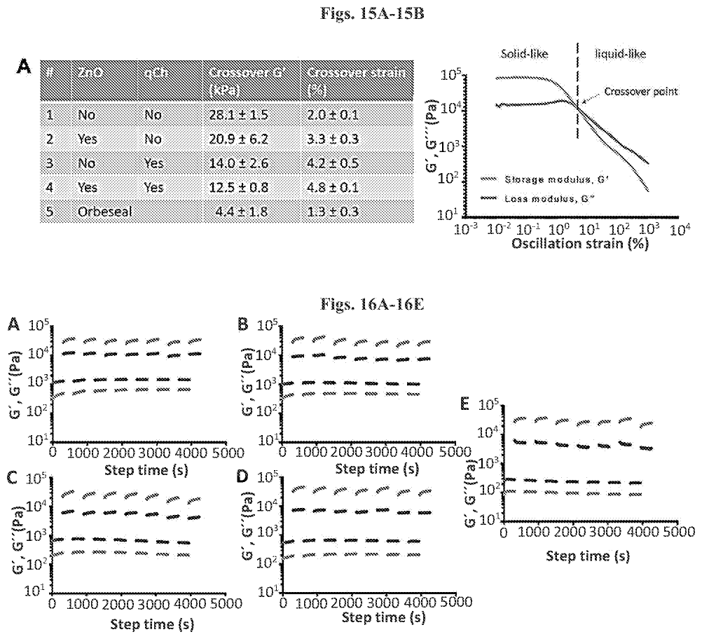

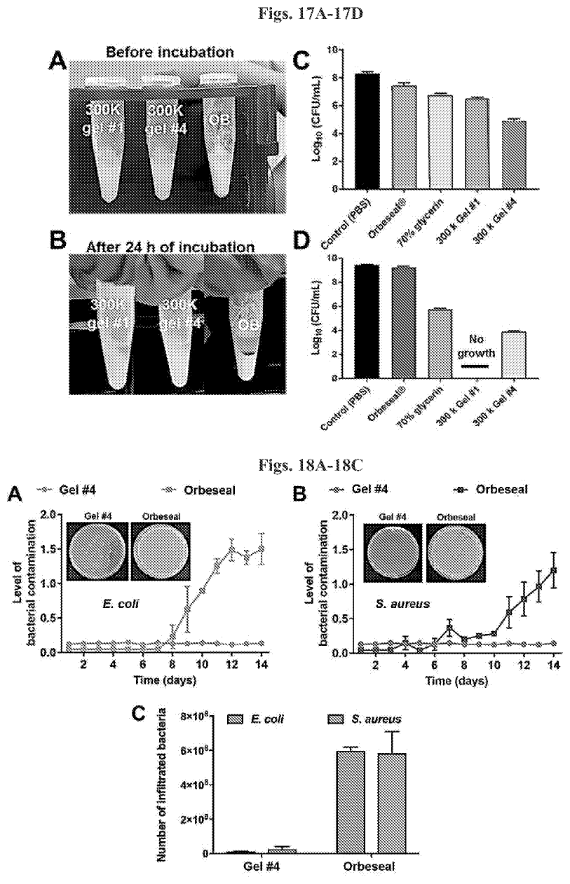

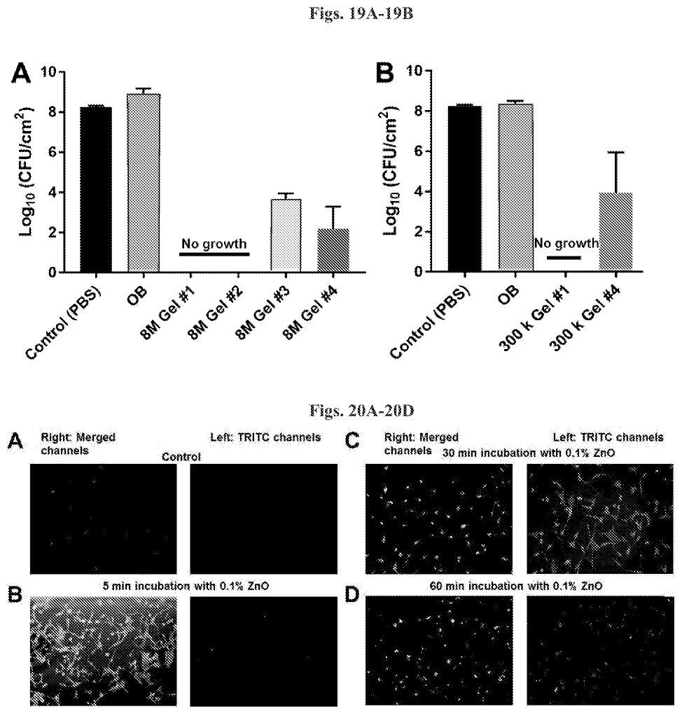

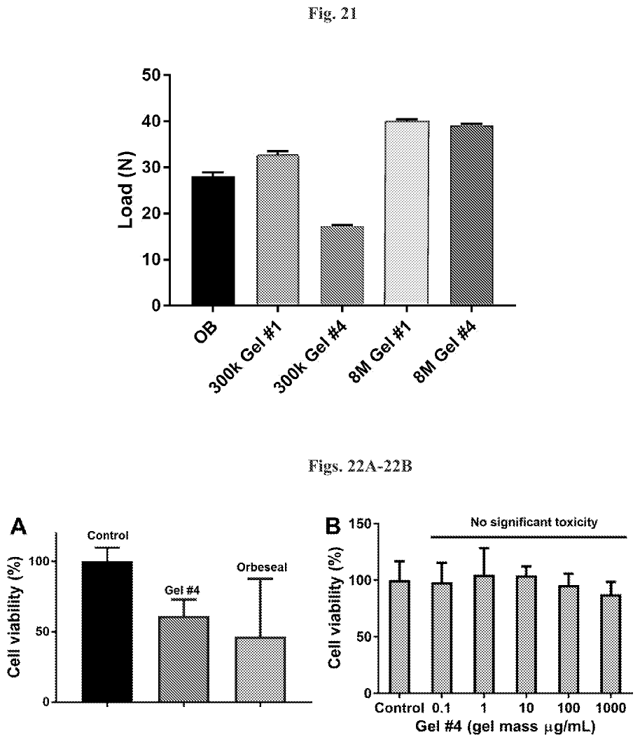

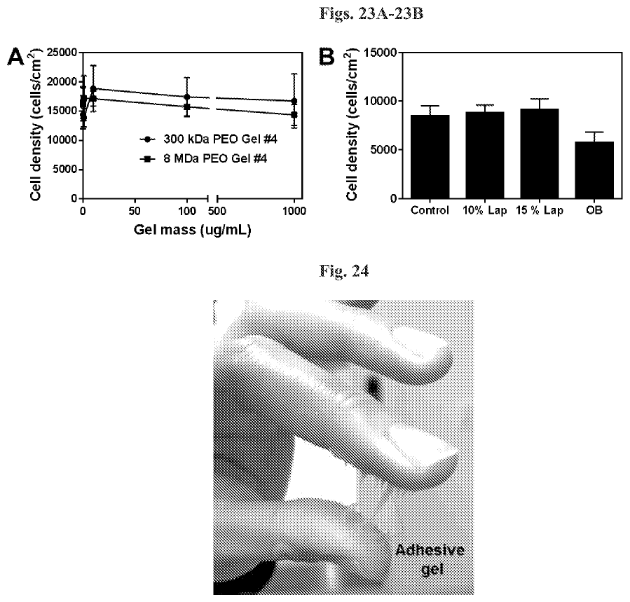

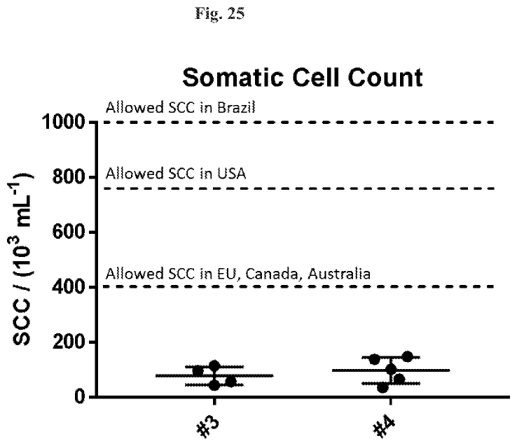

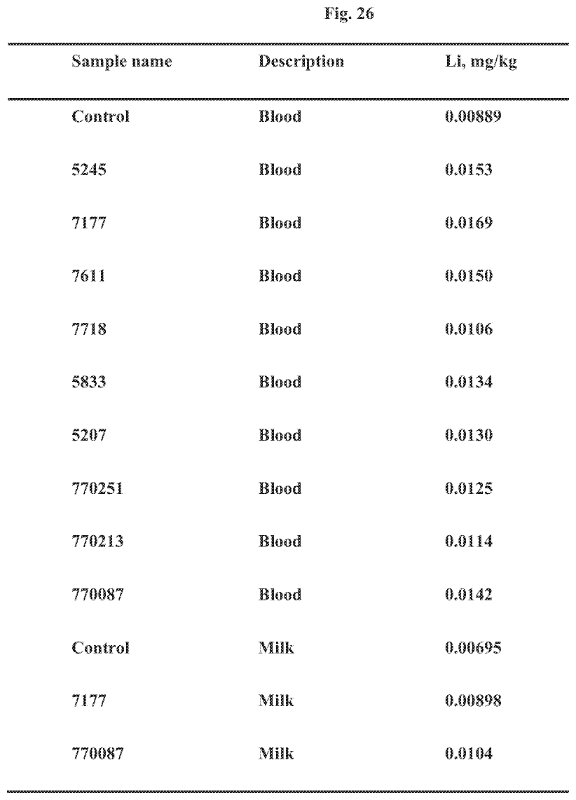

2. After wound creation, manual debridement was performed to completely become dead dermis layer (till see spot bleeding). However, epidermal border (wound edge) which affected by heat (read colour trace of heat signature) was preserved to simulate burn wound more realistically. The gel composition #1-3 from example 13 were applied directly to the wound and spread using sterile spatula, Form dressing made directly contact with the gel formulations. See FIGS. 10-14. Results: The control wound demonstrated infection (all of them), whilst none of the wounds with the gel composition #1-3 did not show any infection. See FIGS. 13-14.

### Example 15—In Vitro Cytotoxicity Study

Initially, the mammalian cell culture was prepared. NIH 3t3 fibroblast cells (86041101, Sigma Aldrich) were cultured in Dulbecco's modified Eagle medium (DMEM; Fisher, Pittsburgh, Pa.) supplemented with 10% newborn calf serum (CS) and 1% penicillin/streptomycin. All cells were cultured to 90% confluence in a 37° C., humidified, 5% CO₂/95% air environment. Cells at passage numbers of 4-7 were used in these experiments.

The cytotoxicity of the various gels with different compositions was investigated using dose escalation and extraction methods as described in ISO 10993-5. For all experiments, Samples were sterilized by exposure to UV radiation for 15 min. In the dose escalation method, respective gel sample was dispersed in sterile Milli-Q water at a concentration of 10 wt % and further serially diluted in cell culture medium to 0.1 to 1,000 μg/ml. On the other hand, in the extraction method, 1 g of each gel sample or commercially available Orbeseal gel was incubated with 5 ml cell culture medium in a conical tube at 37° C. for 48 h to allow for the thorough release of sealant residues. Samples were then centrifuged and cell medium in the supernatant was collected. 3T3 fibroblast cells were seeded on 96-well plates at a cell density of 6,000 cells/cm² (1,950 cells/well) and incubated for 24 h for cell attachment. Next, cells were treated by cell medium with samples as prepared with either dose escalation method or extraction method for designated time intervals. After incubation, samples in cell medium were removed and cells were rinsed twice with PBS. The resulting cell density was determined by PrestoBlue™ Cell Viability Reagent (A13262, Invitrogen). Briefly, the reagent was mixed with cell medium by a volume ratio of 1:10. Cells were then incubated with the reagent/medium mixture for 1 h at 37° C. in a 96-well plate and fluorescence intensity (Em=560 nm/Ex=590 nm) of each sample was measured. To estimate the cell numbers in each well, a standard curve expressing the linear correlation between a range of cell densities and fluorescence intensity (R²>0.95) was plotted, and the cell numbers were determined with this standard curve from the fluorescence intensity recorded in each sample. See FIG. 22B.

### Example 16—Evaluation of Hydrogel Surface Antibacterial Activity

The antibacterial properties of the gels were compared to Orbeseal® Dry Cow Teat Sealant (bismuth subnitrate (65% w/w) in an oil base). Briefly, 0.2 ml of each hydrogel sample or 0.2 ml of Orbeseal were added (in separate wells) into separate 1.5 ml Eppendorf tubes. Samples were then centrifuged and 100 μl of bacterial solution (2*10⁷CFU/ml) was added to each sample. The control samples were bacteria cultured in 0.2 ml of phosphate buffered saline (PBS, pH 7.4). Samples were incubated at 37° C. for 24 h under gentle shaking at 120 rpm. After incubation, samples were resuspended in PBS, serially diluted, and plated on BHI agar plates. The agar plates were then incubated for another 16 hours. Bacterial density on each sample (CFU/cm²) was determined by the number of colonies counted.

In addition, the bacterial viability was also assessed by BacTiter-Glo™ Microbial Cell Viability Assay (Promega, Madison, Wis., USA). Briefly, after incubation, each sample was fully resuspended in PBS followed by further dilution by ten times in PBS. The assay reagent (50 μl) was mixed with an equal volume of each sample on a white opaque 96-well plate. The luminescence of each sample was recorded after incubation for 5 min in room temperature.

### Example 17—Evaluation of the Long-Term Efficacy of Hydrogel Against Bacterial Invasion In Vitro

The ability of gels to prevent bacterial infiltration was evaluated and compared with Orbeseal® Dry Cow Teat Sealant using an in vitro model. gels or Orbeseal® (2 ml) was filled into 5 ml luer-lock syringes and centrifuged at 2000 rpm for 2 min in order to ensure the test materials were free of air bubbles. The syringes were secured in 50 ml conical tubes with their tip ends facing downward. The syringe tips (without needles) were sealed with parafilm during centrifugation. All the syringes with test materials were then sterilized by UV for 15 min and the syringe tip ends were unsealed. Next, 2 ml of bacterial culture of either S. aureus or E. coli (10⁵ CFU/ml) was added to new 50 ml conical tubes and the above prepared syringes were placed downward with their tip ends immersed in the bacterial culture. Additionally, 1 ml of fresh BHI medium was added on top of each test material. The samples were then incubated at 37° C. under 120 rpm shaking. In every 24 h, the bacterial culture in each conical tube was replenished with fresh inoculum. Also, 200 μl of the medium on each sample was withdrawn for OD 600 measurement and bacterial enumeration, followed by the addition of 200 μl of fresh medium. The experiment was carried out for 14 days and each experimental group was triplicated.

### Example 18—Typical Procedure for the In Vivo Safety and Efficacy Study

Efficacy Trials:

Experimental Cows

Holstein cows completing their second lactation will be used in this study. Cows in late lactation (355+/−30 days in milk (DIM) at dry off) producing more than 15 kg of milk each day will be used. The selected cows will have received no parental or intramammary antibiotic or anti-inflammatory treatments within 30 days prior to dry off, have 4 functional quarters with no subclinical or clinical mastitis present on the day of dry off, have teat end scores of less than 3, and have no history of clinical mastitis within the past 30 days (scoring system based on the standard provided by University of Wisconsin). One to 2 weeks before the beginning of the study, the udders of the cows will be examined for milk somatic cell count (SCC), bacteriological culture, and NAGase activity determination. This procedure will be repeated 2 days before the beginning of the study. Cows having a SCC of <200,000 cells/ml of milk, culture-negative milk will be included.

Experimental Groups

The split-herd study design with animal-level treatment allocation will be used and all teats on individual animals will receive the same treatment. Each treatment will be administered on five cows with four teats each. The five experimental groups to be tested in this study are described as follows:
1) 2.6 g of gel formulation #1 (n=5)
2) 2.6 g of gel formulation #2 (n=5)
3) 2.6 g of gel formulation #3 (n=5)
4) 2.6 g of gel formulation #4 (n=5)
5) 2.6 g of Orbeseal teat sealant as the positive control group. (n=5)
6) Untreated cows will serve as the negative control group. (n=5)

Initially a pilot study of 5 animals per group will be performed (25 animals in total). If clinical results are significant, animal numbers could be increased to 40 cows per group.

Administration of Sealant Samples

Treatments are administered by intramammary infusions immediately after the last milking. Prior to each treatment, teats will be cleaned and disinfected with alcohol-based teat wipes to remove solid contaminants. Infusions will be conducted by a small group of trained technicians (presumably one in the pilot study and 10 technicians in the full study). The technicians will be blinded with respect to treatment administration. The sealant will not be massaged into the gland parenchyma after infusion. [1,6,7]

Evaluation of Sealant Stability in the Dry Period

Sealant stability will be evaluated in cows during the dry period by the degree of retention of sealant in the teats during the dry period. Teat-ends will be observed by two trained veterinarians who are blinded to the treatment allocation. Observations are recorded every 2 h for the first 12 hours, daily for the first 2 weeks after infusion, and subsequent observations are made weekly until the end of the dry period at day 60. [6] Sealant retention will be scored on a scale of 0 to 1 with 0 corresponding to no sealant visible at the teat end and 1 corresponding to visible sealant being expressed from the teat end.

At the end of the dry period, the sealant will be squeezed from the teats, placed on filter papers to absorb excessive liquid, and the volume and weight of the remaining sealants will be determined.

Evaluation of Cattle Health During the Dry Period

The incidence of udder inflammation during the dry period, will be assessed by observation every 2 hours for the first 12 hours after infusion, once a day for the following 7 days, and weekly until the end of the dry period at day 60 by two trained veterinarians who will be blinded to the treatment allocation. Inflammation caused by clinical mastitis is scored from 0 to 2 according to the mammary gland chart as follows: 0=normal, 1=abnormal udder (such as swelling, heat, sensitivity, and edema) and 2=abnormal cow (fever, anorexia). Rectal temperature will be determined, and 2 ml of blood sample will be collected daily. [7]

Metabolomic analysis of cow serum in dry period by LC-MS is also conducted to diagnose subclinical mastitis. Five milliliters of blood samples from cows are taken from tail veins at 0, 7, 14, and 21 days after infusion. Serum samples are prepared by centrifuging the blood samples at 1,600 g for 20 min at 4° C. and then stored at −80° C. before use. For metabolomic analysis, metabolites are extracted from serum samples by 1:4 dilution with ethanol/methanol (1:1 v/v). Samples are then vortexed, centrifuged at 16,000 g for 20 min 4° C. and filtered by 0.22 μm membranes. The levels of serum amyloid A, visfatin, and α-tocopherol are analyzed. Metabolomic analysis is performed by injecting 3 μl of each sample into a LC-MS. Metabolites are separated on an Acquity UPLC BEH amide column (2.1 mm×150 mm, 1.7 μm, Waters). Mobile phase A consist of water/acetonitrile (95:5, vol/vol), and mobile phase B is water/acetonitrile (5:95, vol/vol); both mobile phases contain 0.1% formic acid. The elution gradient is 0 min, 99% B; 7.5 min, 40% B; 9 min, 99% B; 10 min, 99% B; and 12 min, 99% B. The flow rate is 0.4 ml/min. The temperature of the column compartment is set to 45° C. The autosampler tray is maintained at 6° C. Sample analysis is performed over a 12-min total run time. [8]

Evaluation of Clinical Mastitis Following Calving

Following calving, clinical mastitis will be recorded daily for 30 days. Clinical mastitis will be defined as no clinical mastitis (0), abnormal milk (1), abnormal milk with inflammation or redness of a quarter (2), or abnormal cow (3) as previously described. Prior to each milking, each quarter shall be observed for these signs by visual inspection of each quarter and the milk obtained during fore-striping using a strip cup. Cows diagnosed with clinical mastitis will be treated at the time of diagnosis according to on-farm protocol.

Measurement of SCC in Milk Following Calving

Somatic cell counts are determined at dry off and again in early lactation. Milk samples (200 ml) are freshly collected from quarters at dry off, 1, 3, 6 and 12, and 30 days in milk (DIM). Milk samples are preserved with a bronopol tablet and refrigerated before SCC testing. Cells are counted manually by a hemocytometer or by flow cytometry. Samples may be diluted by microfiltered skim milk until the SCC obtained is within 100 to 200 cells/μL. Cows diagnosed with subclinical mastitis will be treated at the time of diagnosis by on-farm protocol.

Microbiological Examination in Milk Following Calving

To evaluate the efficacy of gel formulations in reducing the incidence of IMIs compared with either Orbeseal or no product, milk samples (100 μL) collected at the end of the dry period will be plated on 222200 Mastitis SSGN Quad Plate (Eurofins), which allows for visual identification of mastitis-causing organisms by the appearance of colonies, including Staph, Strep, E. coli and Klebsiella species. The milk samples will be streaked on the agar using sterile cotton tipped swabs and incubated at 37° C. for 24 to 48 hours. For those plates having bacterial growth, the identification of bacterial species is recorded. [9]

Milk samples for culture will be collected at dry off, 1, 3, 6, and 12 and 30 days in milk. Additionally, milk samples will be cultured at any time that clinical mastitis is diagnosed.

The cases of intramammary infections are categorized based on microbiological examination results in milk as follows:

AI-IMI: acquired infection during the dry period. At least one organism not present at drying off but identified in a quarter after dry period.

UI-IMI: freshened uninfected during the dry period. An organism not present at drying off and not identified in the same quarter after dry period.

Examination of Laponite Residue in Milk

1.00 ml of milk collected at the end of the dry period, 2.0 ml of 25% tetramethylammonium hydroxide solution, and 2.5 ml of water are added into a PTFE tube. The digestion mixture is pre-treated for 30 min in an ultrasonic bath. Digestion is performed using an ultraCLAVE™ microwave autoclave for pressurized digestions supplied by MLS (Leutkirch, Germany). The microwave power is set at 600 W for 10 min, followed by 30 min at 800 W. The digestion temperature is 140° C. It is kept for approximately 30 min. The amount of lithium in the digest is analyzed using inductively coupled plasma mass spectrometry (ICP-MS) with beryllium added as an internal standard. [10,11, 12]

The lithium concentrations of the milk samples from cows treated with the gels and not treated are both measured using the method above and see if they have a significant difference.

Safety Trials:

Histological Tests

Histological tests will be conducted to evaluate localized inflammatory reactions induced by the sealants. Ten lactating cows producing approximately 7 kg of milk per day and meeting the same health criteria as previously described will be selected for this study. [1] These cows are to be slaughtered after their last lactating period. Sealants will be infused into the teats of each cow after the last milking. After a 60-day dry period, sealant will be removed from the quarters and the cows will be sacrificed. The udders will be collected and separated into two halves after the extraparenchymal tissue has been removed. The streak canal, teat cistern, and udder from each quarter will be collected and processed for histological analysis. Each parenchyma is sampled at the base of the gland adjacent to the gland cistern (zone 1), midway between the gland cistern and dorsal boundary of the mammary parenchyma (zone 2), and near the dorsal parenchymal border (zone 3). [2] Tissues are fixed in 10% neutral buffered formalin for 24 h, processed on an automatic tissue processor and embedded in paraffin wax. Sections will be cut at 4 μm thickness at three levels and stained by Haematoxylin and Eosin (H&E) stain. Additionally, the immunostaining for bovine PMN is conducted in a separate experiment. For the detection of leukocyte adhesion, formalin-fixed tissue sections (5 μm in thickness) will be stained with fluorescein isothiocyanate (FITC)-conjugated mouse anti-bovine CD11b mAb (Serotec, clone CC126 of the IgG2b isotype) by incubating at 4° C. for 60 min. After washed with PBS twice, the tissue samples will be imaged with a confocal fluorescence microscopy. [3]

The degree of inflammation is scored from 1 to 3 based on the levels of polymorphonuclear neutrophilic leukocyte (PMN) infiltration: 1=absence of inflammatory response; 2=moderate PMN infiltration between sealant and tissue interfaces; 3=marked PMN between sealant and tissue interfaces with signs of necrosis. [4]

### REFERENCES

[1] K. R. Petrovski, A. Caicedo-Caldas, N. B. Williamson, Efficacy of a novel internal dry period teat sealant containing 0.5% chlorhexidine against experimental challenge with Streptococcus uberis in dairy cattle, J. Dairy Sci. 94:3366-3375, 2011.
[2] S. C. Nickerson, W. J. Thompson, W. M. Kortum, et al., Histological response of bovine mammary tissue to an intracisternal bead device, J Dairy Sci 70:687-695, 1987.
[3] T. Ozawa, Y. Kiku, M. Mizuno, et al., Effect of intramammary infusion of rbGM-CSF on SCC and expression of polymorphonuclear neutrophil adhesion molecules in subclinical mastitis cows, Vet Res Commun, 36:21-27, 2012.
[4] J. Gogoi-Tiwari, V. Williams, C. B. Waryah, et al., Mammary gland pathology subsequent to acute infection with strong versus weak biofilm forming Staphylococcus aureus bovine mastitis isolates: a pilot study using non-invasive mouse mastitis model, PLOS ONE I DOI:10.1371/journal.pone.0170668, 2017.
[5] S. Godden, P. Rapnicki, S. Stewart, et al., Effectiveness of an internal teat seal in the prevention of new intramammary infections during the dry and early-lactation periods in dairy cows when used with a dry cow intramammary antibiotic, J. Dairy Sci. 86:3899-3911, 2003.
[6] G. H. Lim, K. E. Leslie, D. F. Kelton, et al. Adherence and efficacy of an external teat sealant to prevent new intramammary infections in the dry period. J. Dairy Sci. 90:1289-1300, 2007.
[7] S. Lanctôt, P. Fustier, A. R. Taherian, et al., Effect of intramammary infusion of chitosan hydrogels at drying-off on bovine mammary gland involution, J. Dairy Sci. 100:2269-2281, 2017.
[8] F. Zandkarimi, J. Vanegas, Fern, C. S. Maier, and G. Bobe, Metabotypes with elevated protein and lipid catabolism and inflammation precede clinical mastitis in prepartal transition dairy cows, J. Dairy Sci. 101:5531-5548.
[9] S. Pyorala, L. Kaartinen, and H. Kack, Efficacy of two therapy regimens for treatment of experimentally induced Escherichia coli mastitis in cows, J Dairy Sci 77:453-461, 1994.
[10] S. Hauptkorn, J. Pavel, and H. Seltner, Determination of silicon in biological samples by ICP-OES after non-oxidative decomposition under alkaline conditions, Fresenius J. Anal. Chem. 370: 246-250, 2001.
[11] H. Vanhoe and C. Vandecasteele, Determination of lithium in biological samples by inductively coupled plasma mass spectrometry, Anal. Chim. 244: 259-267, 1991.
[12] D. Saribal. ICP-MS Analysis of Trace Element Concentrations in Cow's Milk Sample from Supermarkets in Istanbul, Turkey, Biol. Trace Elem. Res. 193: 166-173, 2020.

### Example 19—Typical Procedure for the Loading of Agents (e.g. Proteins, Drugs Etc.) onto the Gel

Fabrication of the Gel:

10 g of poly(vinyl acetate) was dissolved in ethyl acetate to make a 100 ml solution for later use. 4.00 g of PEO (20 k molecular weight), 0.400 g of PEO (300 k molecular weight), 44 g of glycerol, and 34 g of water were added to a 600 ml glass beaker. A homogenizer equipped with a PTFE-coated cross impeller was used for stirring. The beaker was put on a hot plate with a thermocouple dipping into the solution for temperature control. The beaker was wrapped with multiple pieces of food wrap to prevent water evaporation. The mixture was heated to 90° C. with 370 rpm stirring. It was kept at 90° C. for 5 min, then allowed to cool down to room temperature. 2.50 g of 20% ZnO nanoparticle aqueous dispersion was added and stirred for 1 min. 15 g of Laponite was added and stirred to mix well. After the gel was formed, it was heated in a 90° C. water bath for 20 min. The gel was cooled to room temperature and then split into two equal parts for the incorporation of bovine serum albumin (BSA) and rhodamine B respectively.

Loading Protein:

Bovine serum albumin (BSA) 125 mg of BSA, 4.75 g of the qCh aqueous solution (contained 0.25 g of qCh and 4.5 g of water), and 5.5 g of glycerol were added, then stirred and mixed with the gel at 320 rpm for 10 min. 6.25 ml of the 10% poly(vinyl acetate) solution and 150 n of acetic acid glacial were added, then stirred at 320 rpm for 7 min before stirring at 2500 rpm for another 10 min.

Loading Drug:

Rhodamine B (Rhodamine B was used as model drug) 4.75 g of the qCh aqueous solution (contained 0.25 g of qCh and 4.5 g of water), and 5.5 g of glycerol were added, then stirred and mixed with the gel at 320 rpm for 10 min. 110 mg of rhodamine B, 6.25 ml of the 10% poly(vinyl acetate) solution, and 150 n of acetic acid glacial were added, then stirred at 320 rpm for 7 min before stirring at 2500 rpm for another 10 min.

Both gels looked very adhesive as it stuck to the beaker at this point. The beakers were wrapped by a piece of food wrap with several small holes poked. It was then placed in a vacuum oven (Eppendorf Vacufuge plus) to evaporate the ethyl acetate and some of the water to strengthen the gel. The setting was 60° C. and “D-AQ.” After 2 h 30 min of evaporation, it was taken out for 1 min of 2500 rpm stirring. 25 ml of PBS was added to each of the beakers. The beakers were put on a plate shaker (Delfia 1296-004) and shaken at the “low” setting for 10 h. The liquid was centrifuged at 21130 rcf for 10 min, and the supernatants were taken for analysis.

### Example 20—the Release Study of Various Agents from the Gel

BSA Percent Release:

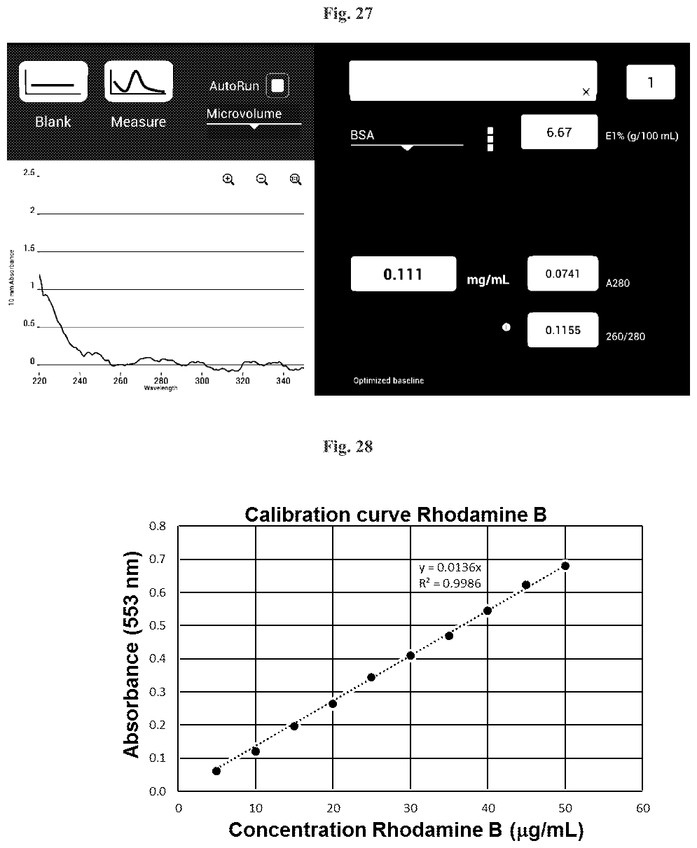

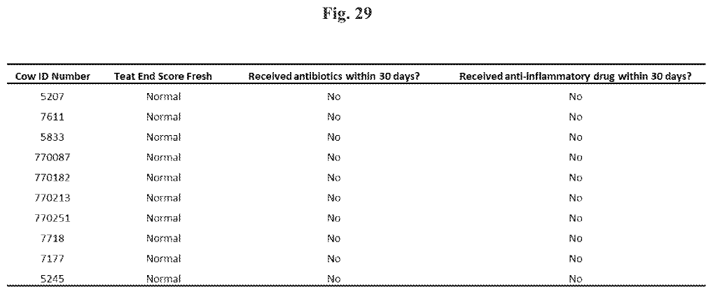

BSA Protein release from the nanoparticles was quantified via nanodrop spectrophotometer to determine the concentration of protein in the supernatant, after creating a nanoparticle pellet on centrifugation, all these after 10 hours of incubation in PBS at room temperature. The release was quantified after 10 hours of incubation in PBS at room temperature a concentration of 0.111 mg/ml of BSA protein was detected in the supernatant of the gels. The amount released corresponds to 2.78 mg and 2.2% release. See FIG. 27.

Rhodamine B Percent Release:

The concentration released of the Rhodamine B was determined by talking the supernatant after 10 h, centrifuged and diluted the solution by 10×. Subsequently, the absorbance was measured by using a plate reader (Varioskan Lux) and compared to a calibration curve. The concentration in supernatant determined was 252 μ/ml, corresponding to 6.3 mg and 5.7% release. See FIG. 28.

### INCORPORATION BY REFERENCE

All U.S. patent application publications and U.S. patents mentioned herein are hereby incorporated by reference in their entirety as if each individual publication or patent was specifically and individually indicated to be incorporated by reference. In case of conflict, the application, including any definitions herein, will control.

Other Aspects

In the claims, articles such as “a,” “an,” and “the” may mean one or more than one unless indicated to the contrary or otherwise evident from the context. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes aspects in which exactly one member of the group is present in, employed in, or otherwise relevant to a given product or process. The invention includes aspects in which more than one, or all of the group members are present in, employed in, or otherwise relevant to a given product or process.

Furthermore, the invention encompasses all variations, combinations, and permutations in which one or more limitations, elements, clauses, and descriptive terms from one or more of the listed claims is introduced into another claim. For example, any claim that is dependent on another claim can be modified to include one or more limitations found in any other claim that is dependent on the same base claim. Where elements are presented as lists, e.g., in Markush group format, each subgroup of the elements is also disclosed, and any element(s) can be removed from the group. It should it be understood that, in general, where the invention, or aspects of the invention, is/are referred to as comprising particular elements and/or features, certain aspects of the invention or aspects of the invention consist, or consist essentially of, such elements and/or features. For purposes of simplicity, those aspects have not been specifically set forth in haec verba herein. It is also noted that the terms “comprising” and “containing” are intended to be open and permits the inclusion of additional elements or steps. Where ranges are given, endpoints are included. Furthermore, unless otherwise indicated or otherwise evident from the context and understanding of one of ordinary skill in the art, values that are expressed as ranges can assume any specific value or sub-range within the stated ranges in different aspects of the invention, to the tenth of the unit of the lower limit of the range, unless the context clearly dictates otherwise.

This application refers to various issued patents, published patent applications, journal articles, and other publications, all of which are incorporated herein by reference. If there is a conflict between any of the incorporated references and the instant specification, the specification shall control. In addition, any particular aspect of the invention that falls within the prior art may be explicitly excluded from any one or more of the claims. Because such aspects are deemed to be known to one of ordinary skill in the art, they may be excluded even if the exclusion is not set forth explicitly herein. Any particular aspect of the invention can be excluded from any claim, for any reason, whether or not related to the existence of prior art.

Those skilled in the art will recognize or be able to ascertain using no more than routine experimentation many equivalents to the specific aspects described herein. The scope of the aspects described herein is not intended to be limited to the above Description, but rather is as set forth in the appended claims. Those of ordinary skill in the art will appreciate that various changes and modifications to this description may be made without departing from the spirit or scope of the invention, as defined in the following claims.

1. A composition, which is a physically reversible gel, comprising
a plurality of silicate nanoparticles;
glycerol;
water;
optionally, a plurality of zinc oxide particles;
a hydrophilic polymer;
a thickening polymer;
a polymer comprising a plurality of quaternary ammonium functional groups; and
an adhesive polymer;
whereby the composition is shear-thinning, viscoelastic and recoverable.

2. A composition, which is a physically reversible gel, consists of
a plurality of silicate nanoparticles;
glycerol;
water;
optionally, a plurality of zinc oxide particles;
a hydrophilic polymer;
a thickening polymer;
a polymer comprising a plurality of quaternary ammonium functional groups; and
an adhesive polymer;
whereby the composition is shear-thinning, viscoelastic and recoverable.

3. A composition, which is a physically reversible gel, comprising
a plurality of silicate nanoparticles;
a hydrophilic polymer;
glycerol; and
water; and
optionally further comprising a thickening polymer; a plurality of zinc oxide particles; a polymer comprising a plurality of quaternary ammonium functional groups and an adhesive polymer; whereby the composition is shear-thinning, viscoelastic and recoverable.

4. The composition according to claim 1, whereby an amount of the plurality of silicate nanoparticles in the composition is from 0.1 wt % to 20 wt %, or 5 wt % to 20 wt %, or 5 wt % to 15 wt %, or 7 wt % to 15 wt %, wherein wt % are percentages of the total weight of the composition.

5. The composition according to claim 3, comprising
1 to 15 wt % plurality of silicate nanoparticles;
0.5 to 10 wt % hydrophilic polymer;
1 to 80 wt % glycerol;
up to 100 wt % water; and optionally further comprising
3 to 9 wt % thickening polymer;
0.2 to 5 wt % plurality of zinc oxide particles;
0.2 to 2 wt % polymer comprising a plurality of quaternary ammonium functional groups; and
0.1 to 50 wt % adhesive polymer;
wherein wt % are percentages of the total weight of the composition.

6. The composition according to claim 1, whereby gelation of the gel is reversed upon addition of a salt.

7. The composition according to claim 1, wherein the silicate nanoparticles are lithium magnesium sodium silicate nanoparticles.

8. The composition according to claim 1, wherein the hydrophilic polymer is selected from the group comprising poly(ethylene oxide), polyvinyl acetate, hydroxypropyl cellulose, and poloxamer.

9. The composition according to claim 8, wherein the hydrophilic polymer is poly(ethylene oxide) having a molecular weight from about 1 kDa to about 10,000 kDa and wherein a 2 wt % solution of the thickening polymer in water at 25° C. has a viscosity from about 200 mPa s to about 10,000 mPa s.

10. The composition according to claim 1, wherein the thickening polymer is selected from the group comprising silicate nanoparticles, lithium magnesium sodium silicate nanoparticles, carboxymethyl cellulose, hydroxypropyl methylcellulose, hydroxyethyl cellulose, and agar, or derivatives thereof.

11. The composition according to claim 1, wherein the polymer comprising a plurality of quaternary ammonium functional groups comprises a plurality of repeat units having the following structure I: 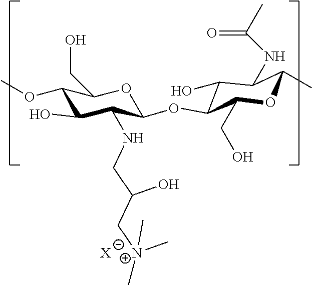
wherein X⁻ is selected from the group comprising Cl⁻, Br⁻, BF₄⁻, and PF₆⁻.

12. The composition according to claim 11, wherein the polymer comprising a plurality of quaternary ammonium functional groups is a chitosan comprising a plurality of quaternary ammonium functional groups.

13. The composition according to claim 1, which further comprises one or more drug or agent.

14. The composition according to claim 13, wherein the one or more drug is selected from the group comprising antibiotic drugs, antiparasitic drugs, antimycotic drugs, analgesics or anti-inflammatory drugs, vitamins, corticosteroid drugs and proteins.

15. The composition according to claim 13, wherein the one or more agent is selected from the group comprising coloring agents or dyes and colorimetric pH indicators.

16. The composition according to claim 1, wherein the composition is in the form a gel having a density from 1.1 g/ml to 5.0 g/ml at about 38° C.

17. The composition according to claim 1 for use in prevention and/or treatment of a disease in a mammal.

18. The composition according to claim 1 for use in prevention and/or treatment mastitis in a mammal.

19. (canceled)

20. A sealant of a duct in a body of a mammal comprising the composition according to claim 1.

21. (canceled)

22. The composition according to claim 1, wherein the composition is administered inside a mammary duct and/or on a skin of an udder.
